(19) United States
(12) Patent Application Publication
(10) Pub. No.: US 2006/0085013 A1
(43) Pub. Date: Apr. 20, 2006

(54) Intraocular lens inserter

(76) Inventors: Vaclav Dusek, Bellevue, WA (US); Jarmila Dusek, Bellevue, WA (US)

Correspondence Address: LAW OFFICES OF RONALD M ANDERSON, 600 108TH AVE, NE, SUITE 507, BELLEVUE, WA, 98004, US

(21) Appl. No.: 10/987,351

(22) Filed: Nov. 12, 2004

Related U.S. Application Data
(63) Continuation-in-part of application No. 10/971,630, filed on Oct. 20, 2004, now abandoned.

(51) Int. Cl. A61F 9/00

(52) U.S. Cl. 606/107

(57) ABSTRACT

An intraocular lens (IOL) inserter and method of use ensure that an artificial intraocular lens is controllably ejected from a cartridge and into a patient's eye by using successive, predefined detent positions. One embodiment has a distal pivotal portion that can be pivoted to a desired angle as a mover advances the IOL into the eye. A detent controls the motion of the mover. In one embodiment, the detent includes a longitudinally extending component with a plurality of stair steps, so that a face of each stair step engages the housing at each different predefined detent position. Other embodiments include at least one orifice in the housing, so that when the orifice is aligned with a pin or protrusion, it engages the orifice to define a detent step. Another embodiment includes a spring-loaded collar that biases a pin into a predefined detent step.

### RELATED APPLICATIONS

This application is a continuation-in-part of a copending patent application Ser. No. 10/971,630, filed on Oct. 20, 2004, the benefit of the filing date of which is hereby claimed under 35 U.S.C. § 120.

### FIELD OF THE INVENTION

The present invention generally relates to a method and ophthalmic surgical devices for implanting lenses, and more specifically, to apparatus and a method for controlling advancement of the intraocular lens from a cartridge and into the eye, so that a lens is advanced through successive, defined steps, which are selected by depressing a detent.

### BACKGROUND OF THE INVENTION

A cataract is a clouding of the normally clear, natural internal lens of the eye. Cataracts cause vision to deteriorate, because the clouding of the lens diffuses light and thus, prevents the light from being focused on the retina, which is required to produce sharp images. This condition is most commonly caused by the natural aging process that causes chemical changes in the natural lens of the eye. Cataracts may also develop as a result of injury to the eye, diabetes, use of steroid medications, previous eye surgery, or an inflammation of the eye. Recent statistics indicate that over one million people undergo cataract surgery each year in the United States, and the majority of these people enjoy substantially improved vision after the surgery.

Intracapsular Cataract Extraction (ICCE) was one technique developed to treat cataracts with a surgical procedure. In this technique, a large incision was made in the eye, because the procedure involved not only removing the entire natural lens, but also removing the entire lens capsule and its attachments within the lens capsule that hold the natural lens in place. Patients were given glasses to wear after this surgery, and they often encountered problems acclimating to the thick lenses required in the glasses. Many patients also needed to remain in bed while the eye healed.

Subsequently, safer surgical techniques were developed that dramatically reduced the recovery time for patients. These new procedures employ an artificial intraocular lens (IOL). The relatively small, lightweight plastic, silicone, or acrylic IOL could be implanted permanently in the eye using a technique known as Extracapsular Cataract Extraction (ECCE). ECCE, unlike ICCE, does not involve removing the lens capsule. Instead, an incision, usually twelve millimeters in length, is made in the eye, and the natural lens is removed in o-e piece. Then, the IOL is inserted into the lens capsule as a replacement for the natural lens. Multiple sutures are required to seal the incision in the eye after this cataract surgery. The IOL thereafter provides the fixed focusing function previously provided by the natural lens, and patients should no longer have need for thick glasses.

Kelman PhacoEmulsification (KPE) is another technique that was developed as a result of searching for ways to perform cataract surgery utilizing a smaller incision. An ultrasound or laser probe breaks the natural lens apart, and the fragments are aspirated from the eye through a three millimeter incision. An IOL is then inserted through this incision, which can be closed with fewer sutures than ECCE, or even allowed to heal unsutured.

The goal of achieving safer cataract surgery and reduced patient recovery time, by using smaller and smaller incisions, continues to drive the development of new material for soft, foldable IOL's and more precise means of implanting the artificial lens. For example, U.S. Pat. No. 4,681,102 (Bartell) discloses apparatus and a method for inserting an IOL through a small incision in an eye, using a load chamber or cartridge in which the IOL is placed. The load chamber is fitted into an injector and both the injector and load chamber are fitted into an insertion cone. A plunger is inserted into the injector and is used to push the IOL from the load chamber into the eye. The IOL unfolds as it emerges from the insertion cone. However, the surgeon cannot readily control the rate at which the lens is ejected from the load chamber and into the eye. Because of variations of IOL material and their diopter, e.g., from 10 diopter to 35 diopter, the mass and stiffness of the IOL affects the “feel” of the IOL as it is advanced into the eye. Thus, a large diopter lens will exhibit a significant friction as it is advanced, and when a surgeon begins to push the IOL out of the load chamber, the surgeon may overshoot the point where the IOL starts to open, because of the sudden release of stored, elastic energy in the IOL. Furthermore, the surgeon does not have the option of rotating a distal portion of the apparatus in order to provide a more comfortable position for the surgeon's hand to utilize the apparatus and thus, provide better control of positioning the IOL through the small incision.

Another prior art IOL insertion device that is disclosed in U.S. Pat. No. 5,582,613 (Brady et al.) utilizes apparatus that includes an inserter with a hand piece and a push rod member. Helical threads are included in a portion of the hand piece, and the threads are adapted to engage and mate with corresponding helical threads on the push rod member so that a surgeon may manually control the rate at which the lens is ejected from the load chamber and into the delicate eye area, by simply varying the rate at which the push rod member is rotated. Although this helical screw assembly provides the surgeon with better control, turning the push rod takes more time and there is no visual indication of the disposition of the IOL in the inserter prior to its ejection from the inserter.

U.S. Pat. No. 5,007,913 (Dulebohn et al.) discloses an apparatus and method for implantation of IOL's that includes a tubular sling that is positionable over parallel working ends of a crossover forceps. The IOL may be folded or curled up and held in position with the forceps tips in a closed position. When the forceps tips are inserted through a small incision in the eye, and opened to release the lens, the lens can be released even though it requires the opening of the forceps tips to a position wider than the incision in the eye. Thus, a controlled release of the lens into the eye is made. However, opening the forceps tips to a position wider than the incision in the eye exposes the patient to a greater risk of injury to the eye if the forceps tips contact delicate parts of the eye. Also, the device is not versatile in that it fails to accommodate commonly used lens injector cartridges by instead utilizing the sling.

Therefore, it would be desirable to provide a device and method that allow full control of advancing and ejecting the IOL as it is ejected from the lens cartridge, provide an adjustable angular orientation of the distal portion of the device, and provide a device and method that are versatile by utilizing proven and commonly used prior art lens cartridges.

### SUMMARY OF THE INVENTION

One aspect of the present invention is directed to an intraocular lens inserter for implanting an artificial lens, preferably of the foldable type, into an eye in a controlled manner. The inserter includes an elongate housing and a mover. The housing has an internal bore having a distal end and a proximal end. The housing also has a plurality of openings. One opening, a cartridge receiver opening, is disposed proximate the distal end of the internal bore and is adapted to receive a cartridge in which an artificial lens is disposed. The cartridge is formed to enable an artificial lens to be forced outwardly therefrom and into an eye. Another opening is disposed at the proximal end of the internal bore.

A mover is sized to slide along a longitudinal axis of the internal bore within the elongate housing and has a distal end that is adapted to pass through the cartridge and force the intraocular lens therefrom and into an eye, and a proximal end adapted to receive a manual force applied by an operator to advance the mover along the longitudinal axis of the internal bore. The mover also includes a detent that interacts with the elongate housing to controllably limit an advancement of the mover through the internal bore, to a plurality of predefined intervals. The detent is biased to engage the elongate housing at the successive predefined intervals. Once thus engaged, further advancement of the mover is precluded by the detent until the detent is released while the mover is advanced. Thus, the intraocular lens is controllably expelled from the cartridge and into an eye by the distal end of the mover as it advances distally through the cartridge.

The mover also includes a push rod joined to a plunger. The push rod, which is preferably substantially smaller in a cross-sectional size than the plunger, is disposed adjacent to the distal end of the internal bore, while the plunger is disposed adjacent to the proximal end of the internal bore. There is also a pin disposed in the plunger, and the pin extends into a groove formed on an inner wall of the elongate housing. The pin and the groove cooperate to prevent the mover from rotating around the longitudinal axis of the internal bore. The elongate housing also includes a first section at its distal end that is pivotally mounted to a second section of the elongate housing, so as to be pivoted to a desired angle relative to the second section. The push rod is sufficiently flexible to readily bend when advancing through the desired angle, to controllably expel the artificial lens from the cartridge.

In one embodiment, an insert is retained inside of the housing and has an inner diameter that defines a cross-sectional size of the internal bore, along which the mover is longitudinally slidable. A groove formed in the insert receives a pin disposed on the plunger. The groove extends along a portion of the longitudinal extent of the insert, so that engagement of the pin by the groove prevents the plunger from being rotated within the internal bore.

The successive predefined intervals controlled by the detent include a first and second detent position. The first detent position limits the push rod advancement to a first point where the intraocular lens has been advanced into a distal portion of the cartridge that is fitted into the inserter. A second detent position limits the push rod advancement to a second point where the intraocular lens is advanced sufficiently so that a portion of the intraocular lens extends from the distal end of the cartridge.

In one embodiment, a spring is disposed in the internal bore, between an intermediate point in the housing and a distal end of the plunger. This spring provides a force that resists advancing the mover distally through the internal bore over at least a portion of its travel, when force is being applied to the mover by the operator to controllably expel the intraocular lens from the cartridge. In one embodiment, the intraocular lens inserter includes a first cavity formed in the plunger for accommodating the detent.

The device also preferably comprises an annular ring disposed around and mounted on the elongate housing, to provide a surface for assisting a user in applying force to the proximal end of the mover while grasping the elongate housing. A surface area is also preferably disposed at the proximal end of the plunger and is disposed such that pressure applied thereto is directed along the longitudinal axis to advance the plunger through the internal bore of the elongate housing.

There are five alternative configurations for the detent. A first configuration comprises a longitudinally extending component having an outer edge, a distal end, and a proximal end. The longitudinally extending component has a plurality of stair steps formed along the outer edge, and each stair step extends outwardly from the first cavity to a different level. A face on each stair step is transverse to the longitudinal axis of the internal bore, and the face abuts against and engages a portion of the elongate housing at one of the successive predefined intervals. However, if the detent is depressed into the first cavity, the face clears its abutment against the elongate housing, so that the mover can be advanced to continue expelling the intraocular lens from the cartridge. The distal end of the longitudinally extending component is mounted to the plunger by a cantilever, so that the plurality of stair steps are biased outwardly from the first cavity by the cantilever. This configuration of the detent can optionally include a spring disposed under the proximal end of the longitudinally extending component so that the spring biases the stairs steps formed on the longitudinally extending component outwardly from the first cavity.

A second cavity is formed in the plunger in this configuration. The detent further comprises a tab formed along an edge that is opposite the edge on which the plurality of stair steps are formed, and the tab extends outwardly from the second cavity and abuts against a portion of the elongate housing to engage the elongate housing prior to the mover reaching one of the successive predefined intervals. The tab serves as insurance; if the plurality of stair steps are depressed excessively, this tab stops an uncontrolled advancement of the push rod by preventing the plurality of stair steps from being depressed into the first cavity by an excessive amount. Excessive deflection of the detent can avoid the stair steps from stopping the advancement of the mover at a successive predefined interval.

The second configuration of the detent comprises a protrusion that extends outwardly from the mover and an orifice is formed in the elongate housing and sized to engage the protrusion at one of the plurality of predefined intervals. The detent also comprises a longitudinally extending component having an outer edge, a distal end, and a proximal end. The protrusion is formed along the outer edge of the longitudinally extending component, extending outwardly from the first cavity, and has a proximal face and a distal face. The distal face of the protrusion abuts against a portion of the elongate housing to engage the elongate housing at each of the plurality of predefined intervals. The protrusion is forcibly disengaged from the portion of the elongate housing when the mover is advanced distally, wherein one portion of the elongate housing comprises the orifice. The distal end of the longitudinally extending component is mounted to the plunger by a cantilever, so that the protrusion is biased outwardly from the first cavity by the cantilever. Alternatively, the longitudinally extending component further includes a disengager, which when activated, disengages the protrusion from the orifice to enable withdrawal of the mover proximally from the housing.

A third configuration of the detent requires a second cavity to be formed in the plunger. The detent then includes a first pin, which is disposed in the first cavity and is biased outwardly therefrom, and a second pin disposed in the second cavity and formed so that the second pin is biased outwardly from the second cavity. An orifice is formed in the elongate housing and is sized to accommodate one of the first pin and the second pin. The first pin is biased to move into the orifice to limit the advancement of the mover through the internal bore at one interval, and the second pin is biased to move into the orifice to limit the advancement of the mover through the internal bore at a successive interval. The detent of this configuration further comprises an arm mounted to the elongate housing. The arm has a detent releaser that is disposed adjacent to the orifice in the elongate housing. The detent releaser is operative, when depressed into the orifice, to move one of the first pin and the second pin out of engagement with the orifice, at successive intervals of advancement of the mover through the internal bore of the elongate housing.

The fourth configuration of the detent has a first orifice, and a second orifice that is longitudinally spaced-apart from the first orifice; both orifices are formed in the elongate housing. The detent in this configuration is disposed in the first cavity and comprises a cantilevered spring arm having a pin disposed on an outer surface of the cantilevered spring arm and sized to fit into engagement within the first orifice and the second orifice, at successive intervals of advancement of the mover through the internal bore of the elongate housing.

The detent of the fourth configuration further comprises an arm mounted to the elongate housing. The arm extends over the first orifice and the second orifice and has a first detent releaser and a second detent releaser that are disposed on its undersurface, adjacent to the first orifice and the second orifice, respectively. The first detent releaser is depressed into the first orifice to move the pin out of engagement with the first orifice, and the second detent releaser is depressed into the second orifice to move the pin out of engagement with the second orifice, at successive intervals of advancement of the mover through the internal bore of the elongate housing.

The fifth configuration of the detent has a second cavity formed in the plunger, and the elongate housing includes an orifice. The detent of this configuration also comprises a collar disposed around the elongate housing, adjacent to the orifice. The collar has a pin disposed on a radially inner surface, adjacent to the orifice and sized to fit within the orifice. A spring is disposed between the exterior of the elongate housing and the radially inner surface of the collar, opposite the pin. The spring biases the pin inwardly through the orifice and into engagement with one of the first cavity and the second cavity at successive intervals of advancement of the mover through the internal bore of the elongate housing. The collar is depressed radially inward, toward the elongate housing, against the bias of the spring, to move the pin out of engagement with one of the first cavity and the second cavity, and thereby to enable the mover to be advanced through the internal bore of the elongate housing between successive steps.

A second aspect of the present invention is directed to a method for implanting an artificial intraocular lens into an eye, by advancing the artificial intraocular lens from a cartridge in a controlled manner. It includes the steps of advancing the artificial intraocular lens through a distal portion of the cartridge until reaching a first step of a detent that prevents further advancement, placing the distal end of the cartridge into the eye; depressing the detent to enable further advancement of the artificial intraocular lens through the distal portion of the cartridge until reaching a second step of a detent that prevents further advancement; and again depressing the detent to enable further advancement of the artificial intraocular lens through the distal portion of the cartridge, until the artificial intraocular lens is fully forced from the distal portion of the cartridge and is fully implanted in the eye.

The method also includes the step of preventing the user from advancing the lens from the cartridge too rapidly, if the user depresses the detent to an extent that would avoid the detent stopping the advancement at the first step of the detent. Also, the artificial intraocular lens is preferably positioned in the cartridge, and the cartridge with the artificial intraocular lens positioned therein is placed adjacent to an opening in the eye, so that the artificial intraocular lens is able to be readily advanced from the cartridge and into the eye in a controlled manner. Another step includes placing the cartridge into a cartridge receiver opening of an internal bore of an elongate housing used for introducing the artificial intraocular lens into the eye from the cartridge. Optionally, the method includes the step of pivoting the cartridge relative to a portion of the elongate housing that is held by a user, to achieve a desired angular orientation of the cartridge when inserting the artificial intraocular lens into the eye.

A third aspect of the present invention is directed to an intraocular lens inserter for implanting an artificial intraocular lens into an eye in a controlled manner. It includes an elongate housing and a mover. The housing includes an internal bore and a handle portion having a distal end and a proximal end disposed along a longitudinal axis. A cartridge receiver portion has a distal end and a proximal end, the cartridge receiver portion being pivotally mounted adjacent to the distal end of the handle portion. A mover is sized to slide within the internal bore of the elongate housing, along the longitudinal axis of the handle portion. The mover has a distal end that is adapted to pass through the cartridge receiver portion and force an artificial intraocular lens therefrom and into an eye, and a proximal end, which is adapted to receive a manual force applied by an operator to advance the mover along the longitudinal axis of the handle portion, to introduce the artificial intraocular lens into an eye.

The mover also includes a plunger at its proximal end and an advancer at its distal end. The advancer is sufficiently flexible so that the advancer readily bends when passing from the handle portion and into the cartridge receiver portion while the cartridge receiver portion is pivoted to an acute angle relative to the handle portion. In one embodiment, a bore reducer is disposed in the internal bore of the handle portion. The bore reducer prevents buckling of the advancer within the handle portion, by confining the advancer to minimize its transverse deflection relative to the longitudinal axis of the handle portion. The intraocular lens inserter preferably includes a surface area disposed at the proximal end of the plunger. The surface area is disposed such that pressure applied thereto is directed along the longitudinal axis to advance the plunger along the longitudinal axis of the handle portion.

In one embodiment, a spring is included to provide a force that resists advancing the mover distally through the elongate housing over at least a portion of a path along which the mover advances through the handle portion, when controllably expelling an artificial intraocular lens from a cartridge.

Optionally, the cartridge receiver portion is selectively pivotal to a desired angular orientation relative to the handle portion, so that it can more readily be employed to introduce an artificial intraocular lens into an eye. In this embodiment, the distal end of the handle portion includes two opposed tabs for pivotally mounting the proximal end of the cartridge receiver portion. The cartridge receiver portion is preferably pivotal relative to the distal end of the handle portion between at least an angular position in which a cartridge is loadable into the cartridge receiver portion, and a desired angular position at which to introduce an artificial intraocular lens into an eye. Thus, the cartridge receiver portion is pivotal relative to the handle portion to form an angle of about 90 degrees, to enable a cartridge to be loaded into the cartridge receiver portion. In a preferred embodiment, the proximal end of the cartridge receiver portion comprises a plurality of adjacent flat surfaces. Each flat surface interacts with the elongate housing for establishing a different angular orientation of the cartridge receiving portion relative to the handle portion. The elongate housing further comprises a sliding block disposed inside the elongate housing adjacent to the distal end of the handle portion and having a distal end and a proximal end. The distal end of the sliding block abuts at least one flat surface of the proximal end of the cartridge receiver portion to bias the cartridge receiver portion at an angular orientation.

A first spring is included to provide a force against the sliding block that advances the sliding block distally of the handle portion and against the cartridge receiver portion, so that the cartridge receiver portion is stabilized at a desired angular orientation relative to the handle portion. A second spring provides a force that resists advancing the mover distally through the housing portion over at least a portion of a path along which the mover advances through the handle portion, and also applies at least a portion of the biasing force applied against the sliding block to bias the sliding block distally of the handle portion and against the cartridge receiver portion.

### BRIEF DESCRIPTION OF THE DRAWING FIGURES

The foregoing aspects and many of the attendant advantages of this invention will become more readily appreciated as the same becomes better understood by reference to the following detailed description, when taken in conjunction with the accompanying drawings, wherein:

FIG. 1A is a cross-sectional side view of a housing for a first preferred embodiment of the lens inserter, which is adapted to receive a first type of an IOL cartridge, and which includes an optional spring component;

FIG. 1B is a cross-sectional side view of an alternative housing for a second preferred embodiment, which is adapted to receive a different type of an IOL cartridge, and which also includes an optional spring component;

FIG. 1C is a cross-sectional top view of a third preferred embodiment having yet another alternative housing that includes a pivotal distal portion, where an IOL cartridge is shown in the pivotal distal portion in phantom view;

FIG. 1D is a cross-sectional side view of the housing of FIG. 1B with an optional proximal orifice for a fourth preferred embodiment;

FIG. 2A is a top view of the housing for the first preferred embodiment shown in FIG. 1A;

FIG. 2B is a top view of the housing for the second preferred embodiment shown in FIG. 1B;

FIG. 2D is a top view of the housing for the fourth preferred embodiment shown in FIG. 1D;

FIG. 3A is a side view of the prior art IOL cartridge that is accommodated by the housing shown in FIGS. 1A and 2A;

FIG. 3B is a side view of the different prior art IOL cartridge that is accommodated by the housing shown in FIGS. 1B and 2B;

FIG. 4A is a cross-sectional side view of a configuration of a mover that has a cantilevered multi-step detent and is usable in either the first, second, or fourth embodiment;

FIG. 4B is a cross-sectional side view of a configuration of a mover that has a helical spring-loaded, multi-step detent and is usable in either the first, second, or fourth embodiment;

FIG. 4D is a cross-sectional side view of a configuration of a mover that has a second configuration of a multi-step detent that is cantilevered and is usable in either the first, second, or fourth embodiment;

FIG. 4E is a cross-sectional side view of a configuration of a mover that has the cantilevered multi-step detent with a disengager and which is usable in either the first, second, or fourth embodiment;

FIG. 5 is a cross-sectional side view of the third preferred embodiment of an assembled intraocular lens inserter with a phantom view of the pivotal distal portion and the prior art IOL cartridge (that is used with the first embodiment) and pivotal distal portion of the housing pivoted to a position for loading/unloading the IOL cartridge;

FIG. 6A is a cross-sectional side view of the configuration of the mover with the cantilevered multi-step detent of FIG. 4A shown in a first detent position relative to the housing of FIG. 1A;

FIG. 6B is a cross-sectional side view of the configuration of the mover with a spring-loaded multi-step detent of FIG. 4B, shown in a first detent position relative to the housing of FIG. 11B;

FIG. 6C is a cross-sectional side view of the third preferred embodiment of an assembled intraocular inserter device with the housing having the pivotal distal portion, wherein an alternative configuration of a mover has a flexible advancer and is shown in the first detent position, and a prior art IOL cartridge is shown in phantom view in the pivotal distal portion;

FIG. 6D is a cross-sectional side view of the configuration of the mover with the cantilevered multi-step detent of FIG. 4D shown in a first detent position relative to the housing of FIG. 1D;

FIG. 6E is a cross-sectional side view of the configuration of the mover with the cantilevered multi-step detent and with the disengager of FIG. 4E, where the multi-step detent is shown in a first detent position relative to the housing of FIG. 1D;

FIG. 7A is a cross-sectional side view of the configuration of the mover with the cantilevered multi-step detent shown in a second detent position relative to the housing of FIG. 1A;

FIG. 7B is a cross-sectional side view of the configuration of the mover with the helical spring-loaded multi-step detent shown in a second detent position relative to the housing of FIG. 1B;

FIG. 7C is a cross-sectional side view of the third preferred embodiment of an assembled intraocular inserter device with a pivotal distal portion, wherein the mover with the flexible advancer is shown with the cantilevered multi-step detent in the second detent position, and a prior art IOL cartridge is shown in phantom view in the pivotal distal portion of the housing;

FIG. 7D is a cross-sectional side view of the configuration of the mover with the cantilevered multi-step detent shown in a second detent position relative to the housing of FIG. 1D;

FIG. 7E is a cross-sectional side view of the configuration of the mover with the cantilevered multi-step detent having the disengager, shown in a second detent position relative to the housing of FIG. 1D;

FIG. 8A is a cross-sectional side view of the configuration of the mover with the cantilevered multi-step detent shown with the mover in a fully advanced position relative to the housing of FIG. 1A;

FIG. 8B is a cross-sectional side view of the configuration of the mover with the helical spring-loaded multi-step detent shown with the mover in a fully advanced position relative to the housing of FIG. 1B;

FIG. 8C is a cross-sectional side view of the third preferred embodiment of an assembled intraocular inserter device having the housing with the pivotal distal portion, wherein the mover with the flexible advancer is shown in a fully advanced position and the prior art IOL cartridge is shown in phantom view in the pivotal distal portion, which is selectively set at an acute angle;

FIG. 8D is a cross-sectional side view of the configuration of the mover with the cantilevered multi-step detent, shown with the mover in a fully advanced position relative to the housing of FIG. 1D;

FIG. 8E is a cross-sectional side view of the configuration of the mover with the cantilevered multi-step detent and the disengager, shown with the mover in a fully advanced position relative to the housing of FIG. 1D;

FIG. 9 is a cross-sectional side view of a portion of the third preferred embodiment of an assembled intraocular inserter device having the housing with the pivotal distal portion that includes indexing means comprising protrusions that index with a notch;

FIG. 10 is a cross-sectional side view of a portion of the third preferred embodiment of an assembled intraocular inserter device having the housing with the pivotal distal portion that includes indexing means comprising notches that index with a protrusion;

FIG. 11 is a cross-sectional side view of a portion of the third preferred embodiment of an assembled intraocular inserter device having the housing with the pivotal distal portion that includes indexing means comprising notches that index with a ball biased with a spring (not shown);

FIG. 12A is a top view of a portion of the third preferred embodiment of an assembled intraocular inserter device having the housing with the pivotal distal portion that includes indexing means comprising two balls biased with springs (not shown) indexing with indentations;

FIG. 12B is a cross-sectional side view of a portion of the third preferred embodiment shown in FIG. 12A;

FIG. 13A is a cross-sectional side view of a fifth preferred embodiment of an inserter with a third configuration of a multi-step detent shown prior to advancement of a mover in which it is included, along the longitudinal axis of the housing, to a first detent position;

FIG. 13B is a cross-sectional side view of the fifth preferred embodiment of an inserter with the third configuration of the multi-step detent of FIG. 13A, after the mover has been advanced to the first detent position;

FIG. 13C is a cross-sectional side view of the fifth preferred embodiment of an inserter with the third configuration of the multi-step detent of FIGS. 13A and 13B, after the mover has been advanced to a second detent position;

FIG. 14A is a cross-sectional side view of a sixth embodiment of an inserter with a fourth configuration of the multi-step detent, shown prior to advancement of a mover, along the longitudinal axis of the housing, to a first detent position;

FIG. 14B is a cross-sectional side view of the sixth embodiment of an inserter with the fourth configuration of the multi-step detent FIG. 14A, after the mover has been advanced to the first detent position;

FIG. 14C is a cross-sectional side view of the sixth embodiment of an inserter with the fourth configuration of the multi-step detent of FIGS. 14A and 14B, after the mover has been advanced to a second detent position;

FIG. 15A is a cross-sectional side view of a seventh embodiment of an inserter with a fifth configuration of the multi-step detent shown prior to advancement of a mover, along the longitudinal axis of the housing, to a first detent position;

FIG. 15B is a cross-sectional side view of the seventh embodiment of an inserter with the fifth configuration of the multi-step detent of FIG. 15A, after the mover has been advanced to the first detent position; and

FIG. 15C is a cross-sectional side view of the seventh embodiment of an inserter with the fifth configuration of the multi-step detent of FIGS. 15A and 15B, after the mover has been advanced to a second detent position.

### DESCRIPTION OF THE PREFERRED EMBODIMENT

### Applicability of the Present Invention

The present invention provides a versatile and improved apparatus and method for inserting an artificial IOL into an eye during surgery. The apparatus facilitates a safer procedure for accomplishing this function, because it enables a controlled advancement of the artificial IOL into an eye, through successive predefined steps, thereby minimizing inadvertent uncontrolled or premature release of the lens from the distal end of a cartridge. One embodiment of this invention also enables a user to selectively adjust the distal portion of the inserter at a desired angle that aids the user by providing a more comfortable hand position in which to use the apparatus and thus, better control positioning of the artificial IOL in the eye.

Each embodiment of the intraocular lens inserter includes a housing, a mover, and a multi-step detent. Two configurations of the housing are each configured to receive a different type of prior art IOL cartridge commonly employed for introducing an artificial IOL into the eye, and a third embodiment (which can otherwise be configured to accept any common IOL cartridge) has a housing that includes a distal portion that can be selectively set at a desired angle by a user. Each configuration of the mover is moved along the longitudinal axis of the housing between successive predefined points that are defined by a configuration of the multi-step detent. By limiting the advancement of the mover and corresponding advancement of the IOL from the distal end of the IOL cartridge, the multi-step detent ensures that a user controllably ejects the artificial IOL from the IOL cartridge into the eye, preventing an inadvertent uncontrolled ejection. The multi-step detent also provides a clear visual indication of the disposition of the artificial IOL as it travels along the longitudinal axis of the IOL cartridge prior to ejection into the eye.

While it is particularly useful for implanting IOLs during surgery to treat cataracts, it must be emphasized that the present invention is neither limited to cataract surgery nor limited to use during ocular surgery on human patients. For example, the present invention can be utilized in any eye surgery that requires controlled ejection of either an artificial IOL or similar lens, and can readily be adapted to control the ejection of other medical devices from inside a cartridge or other temporary holding device. Furthermore, the present invention is applicable to surgical procedures performed on animal patients, such as dogs that may require cataract surgery.

The present invention is also versatile in that its housing can readily be adapted to accommodate any cartridge into which an artificial IOL (or other medical device) is loaded prior to use of the present invention to insert the IOL or device into a patient's eye or other portion of the body. Thus, it must also be emphasized that the present invention is not limited to use with the specific prior art cartridges as shown in FIG. 3A (which is described in U.S. Pat. No. 5,582,613 assigned to Allergan) and FIG. 3B (which is described in U.S. Pat. No. 6,083,231 assigned to Alcon Laboratories, Inc.). These cartridges are shown in connection with this invention, because they are two of the type that are often used for IOL insertion by ocular surgeons.

### First, Second, Third, and Fourth Preferred Embodiments of Intraocular Lens Inserter

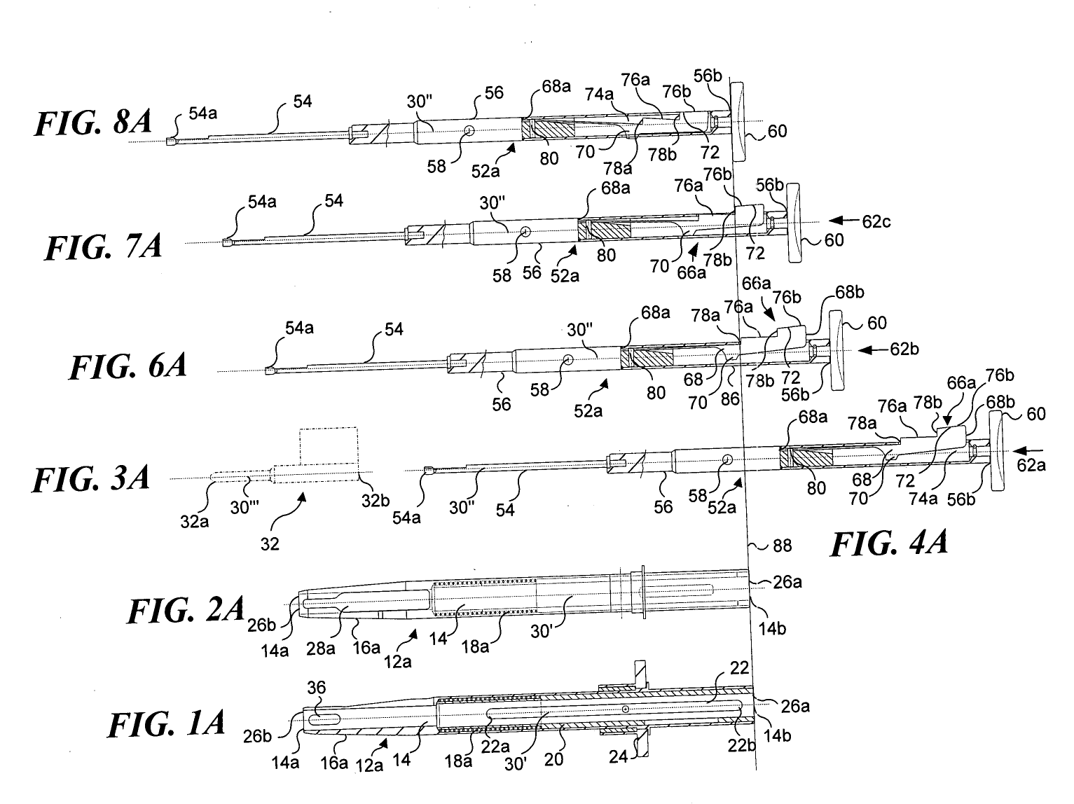

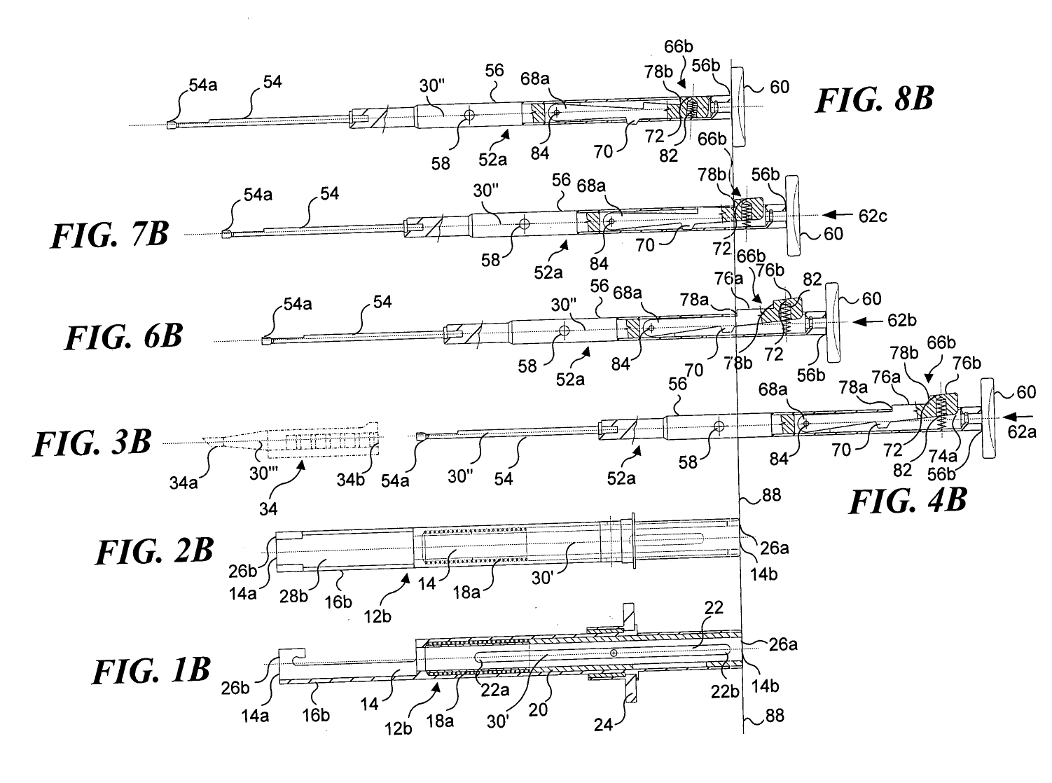

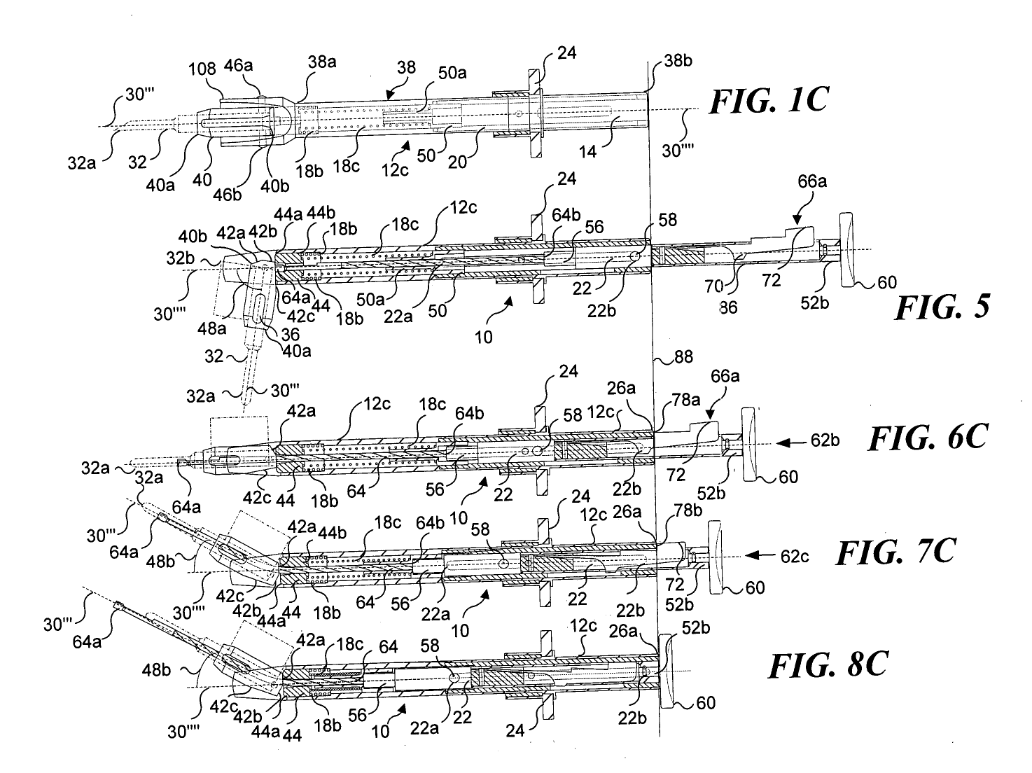

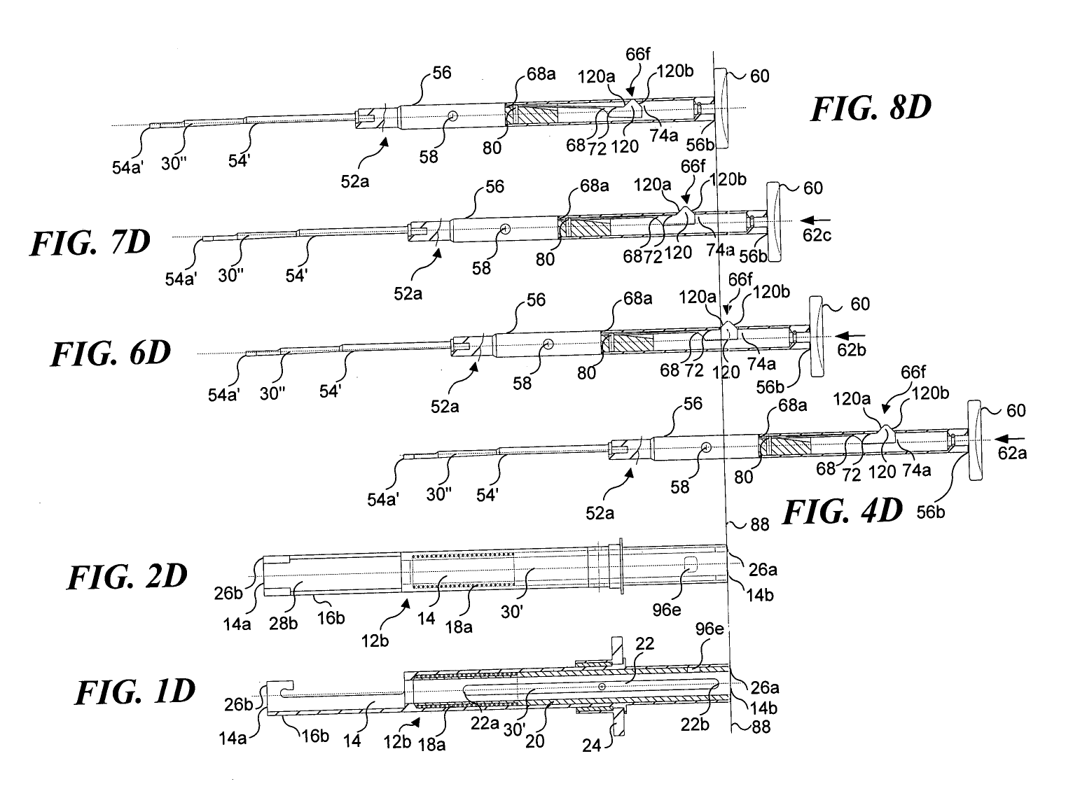

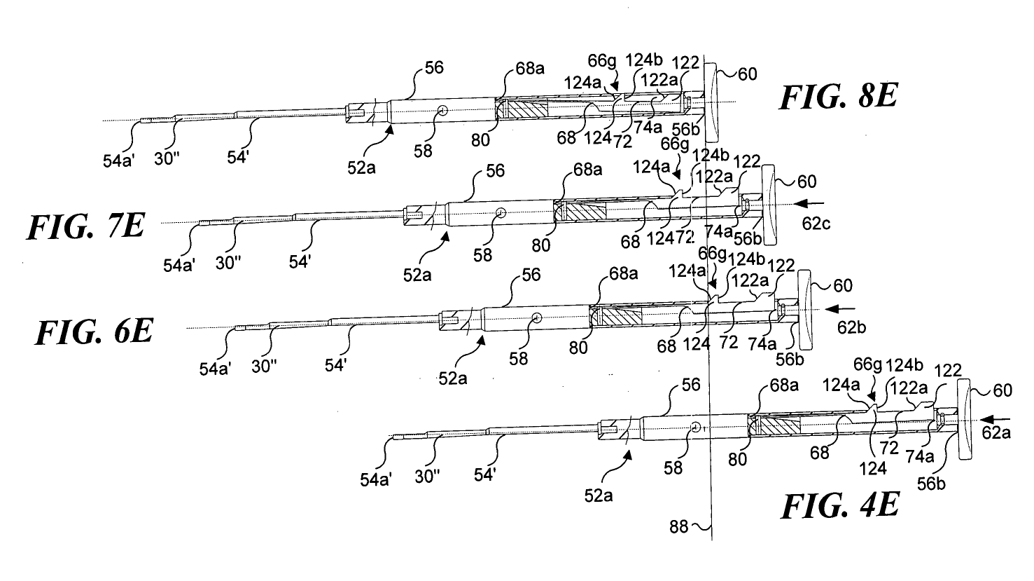

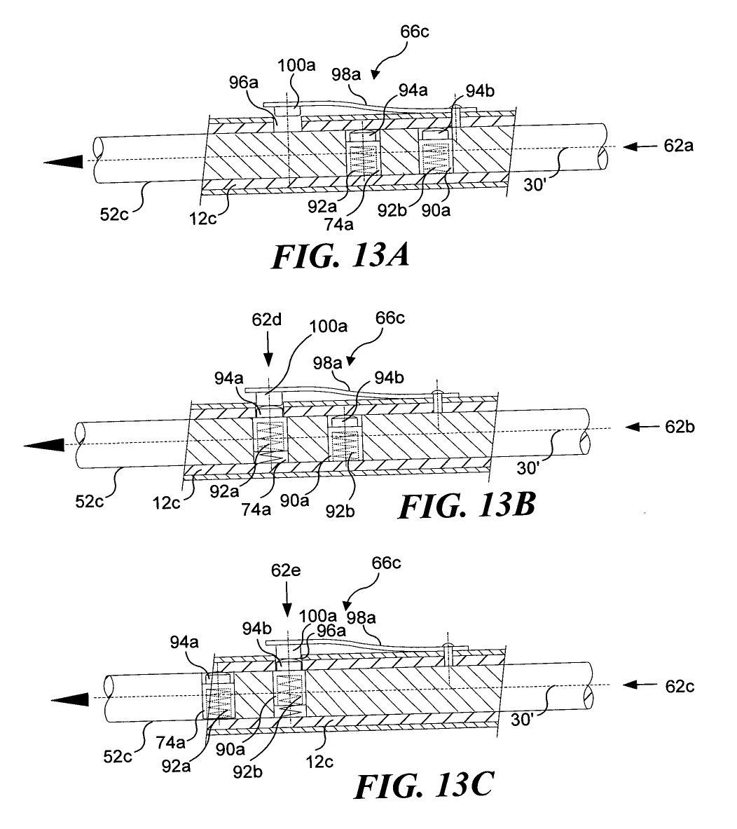

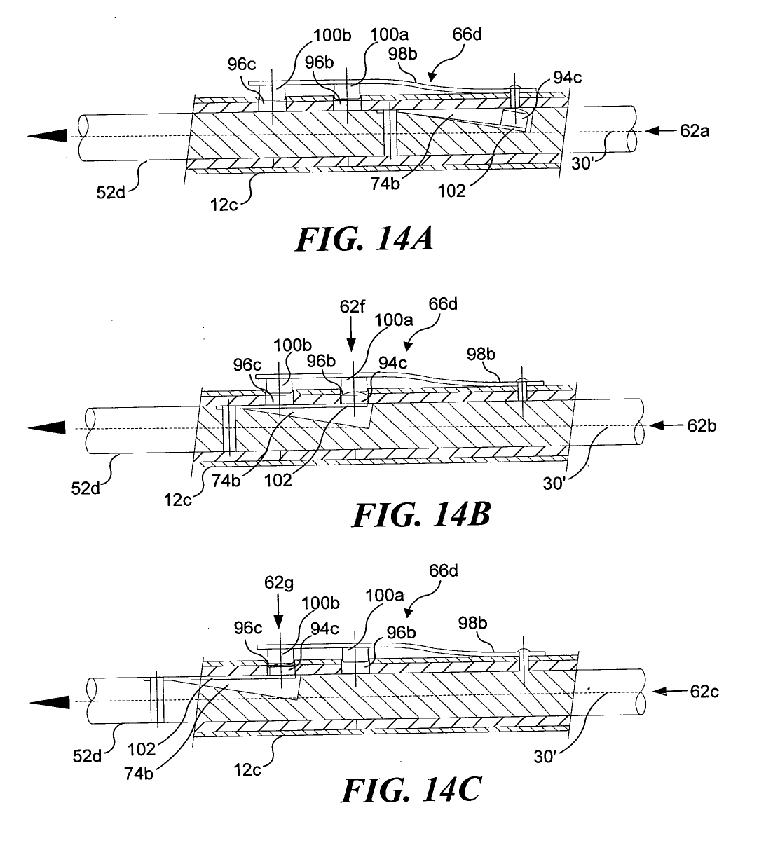

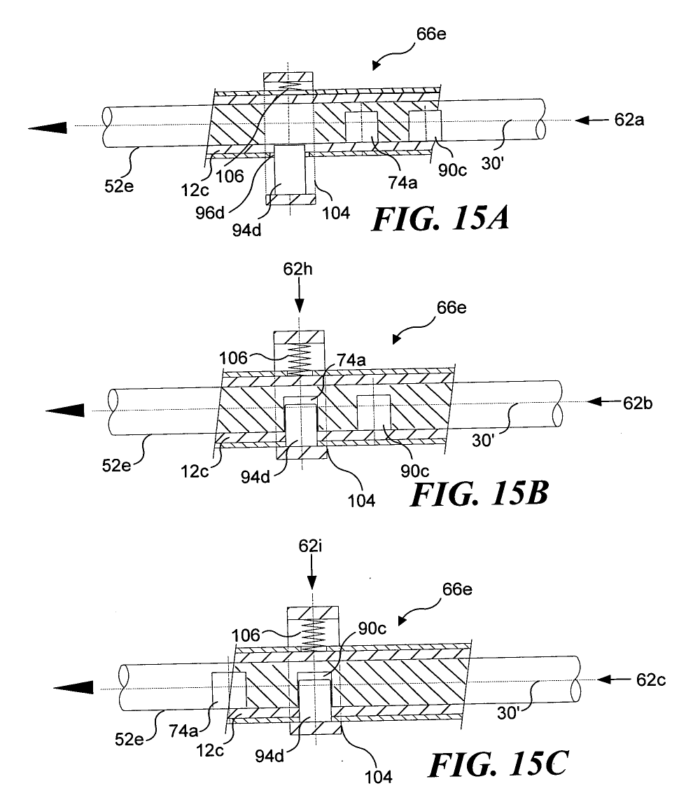

While not limited to the disclosed preferred embodiments, the following discussion describes details of several different configurations of the intraocular lens inserter, including three housing configurations, two mover configurations, and five multi-step detent configurations. By way of example, a complete IOL inserter can comprise a housing 12a as shown FIG. 1A, or a housing 12b as shown FIG. 1B, or a housing 12c as shown FIG. 1C, or a housing 12b with an optional proximal housing orifice as shown in FIG. 1D. Similarly, a complete IOL inserter can include a mover 52a as shown in FIG. 4A, or a mover 52b, as shown in FIG. 5, as well as either a cantilevered multi-step detent 66a of FIG. 4A, or a spring-loaded multi-step detent 66b of FIG. 4B, or a cantilevered multi-step detent 66f of FIG. 4D, or a cantilevered multi-step detent 66g with a disengager of FIG. 4E, or a multi-step detent 66c, shown in FIGS. 13A, 13B, and 13C, or a multi-step detent 66d, as shown in FIGS. 14A, 14B, and 14C, or a multi-step detent 66e, shown in FIGS. 15A, 15B, and 15C. A prior art IOL cartridge 32 having a distal end 32a and a proximal end 32b, as shown in FIG. 3A, can be used with housing 12a (FIG. 1A), while a different prior art IOL cartridge 34 having a distal end 34a and a proximal end 34b, as shown in FIG. 3B can be used with housing 12b (FIG. 1B or FIG. 1D). As discussed above, the prior art IOL cartridges are not part of the present invention but are illustrated to demonstrate the versatility of the apparatus in accommodating various different IOL cartridge designs and to demonstrate how the present invention is able to controllably expel the artificial IOL from these different types of prior art IOL cartridges.

For purposes of clarity, mover 52a, housing 12a, and prior art IOL cartridge 32 are shown separately in FIG. 4A, FIG. 1A, and FIG. 3A, respectively, in a first preferred embodiment. Likewise, mover 52a, housing 12b, and different prior art IOL cartridge 34 are also shown separately in FIG. 4B, FIG. 1B, and FIG. 3B, respectively, in a second preferred embodiment. Similarly, mover 52a, housing 12b with optional proximal orifice, and different prior art IOL cartridge 34 are also shown separately in FIG. 4D, FIG. 1D, and FIG. 3B, respectively, in a fourth preferred embodiment. Those skilled in the art will recognize that although prior art IOL cartridge 32 cannot be used with housing 12b and that different prior art IOL cartridge 34 cannot be used with housing 12a, the housing of the present invention is readily configured to accommodate alternative designs of IOL cartridges. Furthermore, it should be noted that mover 52a may be used with housing 12a or housing 12b. In addition, cantilevered multi-step detent 66a may be used with any of the housing configurations and spring-loaded multi-step detent 66b may be used with any of the housing configurations. Likewise, cantilevered multi-step detent 66f and cantilevered multi-step detent 66g with the disengager may be used with any of the housing configurations, provided that the proximal orifice is included with the housing. However, due to the selectively adjustable angular feature of housing 12c that is achieved by providing the pivotal portion at the distal end of the housing, mover 52b is best used with housing 12c, since mover 52b includes a flexible portion in order to readily bend around the result curve as the mover is advanced with the pivotal portion set to a selected acute angle relative to the rest of housing 12c, such as complete IOL inserter 10 shown in FIG. 7C, in a third preferred embodiment

FIG. 6A, FIG. 7A, and FIG. 8A illustrate only mover 52a and respectively show cantilevered multi-step detent 66a in a first detent position, a second detent position, and a fully advanced position, as described below. Similarly, FIG. 6C, FIG. 7C, and FIG. 8C illustrate mover 52b in housing 12c, and respectively illustrate the cantilevered multi-step detent in the first detent position, the second detent position, and the fully advanced position, as well as illustrating the corresponding angular orientation at which the distal portion of the housing typically will be positioned at each successive predefined interval. However, those skilled in the art will realize that the present invention is not limited to the detent positions shown in the Figures and described below, and that additional detent positions can readily be provided.

### Housing Configurations

Housing 12a is generally elongate in shape, as shown in the side view of FIG. 1A and top view of FIG. 2A. Similarly, mover 52a is also elongate in shape, as shown in FIG. 4A, and cantilevered multi-step detent 66a is mounted at the proximal end of this mover, also as shown in FIG. 4A. In addition, the distal end of the housing also is configured to accommodate an IOL cartridge, such as prior art cartridge 32, which is shown in FIG. 3A.

Returning to FIG. 1A, housing 12a includes an internal bore 14 that comprises a distal end 14a and a proximal end 14b. The cross-sectional size of the internal bore is defined at least in part by an inner diameter of an insert 20. The housing also includes a distal portion 16a, a helical spring 18a that provides a biasing force resisting advancing of the mover through the internal bore over at least part of its travel, a groove 22 that is formed in insert 20, an annular ring 24 that is rotatable around the exterior of the housing to provide a surface for supporting a user's fingers, a proximal opening 26a disposed at the proximal end of the housing, a distal opening 26b disposed at the distal end of the housing, a cartridge receiver opening 28a, (FIG. 2A) and a longitudinal axis 30′. Cartridge receiver opening 28a (FIG. 2A) is also disposed at the distal end of housing 12a. Distal portion 16a is sized so that cartridge receiver opening 28a will accommodate prior art IOL cartridge 32 (shown in FIG. 3A). In its assembled form (not shown), a distal end 32a of prior art IOL cartridge 32 (FIG. 3A) is inserted through cartridge receiver opening 28a (FIG. 2A) and the IOL cartridge is positioned such that distal end 32a protrudes from distal opening 26b in the housing. A longitudinal axis 30′″ of the prior art cartridge shown in FIG. 3A is then aligned with the longitudinal axis 30′ (FIG. 1A) of the housing. Note that an opening 36 (FIG. 1A) is optionally included in the housing to enable a user to view the disposition of the mover within the prior art cartridge. When the inserter is used, the IOL cartridge is folded open, an artificial IOL is folded within the prior art cartridge, the IOL cartridge is closed with the lens inside, and the prior art IOL cartridge and lens are inserted into the cartridge receiver opening. The IOL cartridge is designed so as to enable the artificial IOL to be forced through and outwardly from its distal end 32a, (FIG. 3A) and into an eye.

Turning back to FIG. 1A, groove 22, which is formed in insert 20, engages a pin 58 (FIG. 4A) on mover 52a, and the groove and pin cooperate to limit the longitudinal motion of the mover to a predefined range, extending from a proximal end 22b of groove 22, to a distal end 22a of the groove. Engagement of pin 58 (FIG. 4A) in groove 22 (FIG. 1A) also prevents the mover from rotating about longitudinal axis 30′ of housing 12a, within the housing. Optional helical spring 18a is disposed proximate to the distal portion of the housing so as to provide a bias force that resists the mover advancing distally along the longitudinal axis of the housing, particularly as the mover approaches the distal limit of its travel. Annular ring 24 is disposed circumferentially around the housing and, as noted above, provides a surface that assists the user in grasping and holding the housing steady as the mover is advanced.

Housing 12b is shown in FIG. 1B and is similar in all respects to housing 12a (FIG. 1A) except that it accommodates different type of prior art IOL cartridge 34, which is shown in FIG. 3B. Thus, a distal portion 16b in housing 12b is different in configuration than distal portion 16a of housing 12a, because the housing 12b has a cartridge receiver opening 28b that is formed to accommodate the shape of the different prior art IOL cartridge as shown in FIG. 3B. Furthermore, housing 12b as shown in FIG. 1D includes a proximal orifice 96e to accommodate either cantilevered multi-step detent 66f or cantilevered multi-step detent 66g that includes a disengager 122.

Housing 12c, which is shown in FIG. 1C, includes an internal bore 14, and a pivotal portion of the housing includes a cartridge receiver portion 40 having a distal end 40a and a proximal end 40b (FIG. 5). The pivotal portion of the housing is pivotally mounted to a handle portion 38 (FIG. 1C), which includes a distal end 38a and a proximal end 38b. Thus, housing 12c allows the user to selectively adjust the angle of the pivotal portion relative to the handle portion, to facilitate more precise maneuverability and control of the inserter. In addition, the cartridge receiver portion is readily angled substantially at a right angle to the handle portion of the housing to facilitate loading prior art IOL cartridge 32 (FIG. 3A) into cartridge receiver portion 40.

The pivoting portion is mounted to a fork 108 comprising two opposed tabs 46a and 46b that are disposed at the distal end of the handle portion, as shown in FIG. 1C. As shown in FIG. 5, to load the prior art IOL cartridge into the cartridge receiver portion, the cartridge receiver portion is pivoted relative to fork 108 and a longitudinal axis 30″″ of the handle portion until an angle 48a formed between longitudinal axis 30″″ of the handle portion and longitudinal axis 30′″ of the pivotal portion is approximately ninety degrees. Moreover, as shown in FIG. 8C, during use of the inserter to insert an artificial IOL into the eye, the surgeon may selectively position the cartridge receiver portion at a smaller acute angle 48b.

To ensure that the pivotal portion remains at the desired acute angle that was selected relative to the handle portion, cartridge receiver portion 40 can be configured with a variety of indexing means. Indexing means includes any type of structure that causes the cartridge receiver portion to be retained at a desired position relative to the handle portion, until moved as desired to a different position. For example, cartridge receiver portion 40 includes a plurality of angled flat surfaces 42a (FIG. 5), 42b (FIG. 5), and 42c (FIG. 8C) that interact with a sliding block 44 having a distal end 44a and a proximal end 44b. As shown in FIG. 5, a helical spring 18b and a helical spring 18c together apply a biasing force against sliding block 44 (FIG. 5) that urges the sliding block against one of the angled flat surfaces with sufficient force to provide resistance against the angled portion inadvertently moving to a different angled flat surface. Most of the biasing force for this purpose is provided by helical spring 18b, since helical spring 18c has a substantially smaller spring constant and is intended to provide the much lower biasing force that resists the distal advancement of the mover through internal bore 14 (FIG. 1C). Sliding block 44 is disposed inside housing 12c and adjacent to the distal end of the handle portion. When the user wants to load the IOL cartridge into the cartridge receiver portion, the pivotal portion is pivoted so that angled flat surface 42c abuts against distal end 44a of the sliding block, as shown in FIG. 5. Because the sliding block exerts a force against angled flat surface 42c (FIG. 5), the pivotal portion resists being moved unintentionally to a different angled position relative to the handle portion.

When the user wants to pivot the cartridge receiver portion to angle 48b, as shown in FIG. 8C, the pivotal portion is pivoted such that adjacent flat surface 42a abuts against distal end 44a of the sliding block. Helical spring 18b and helical spring 18c again provide the total biasing force acting on the sliding block to stabilize the cartridge receiver portion at angle 48b. Those skilled in the art will recognize that the number of angled flat surfaces that may be formed on the proximal end of the pivotal portion can readily be increased or decreased in order to provide either more or fewer discrete positions at which the pivotal portion of the housing can be selectively set.

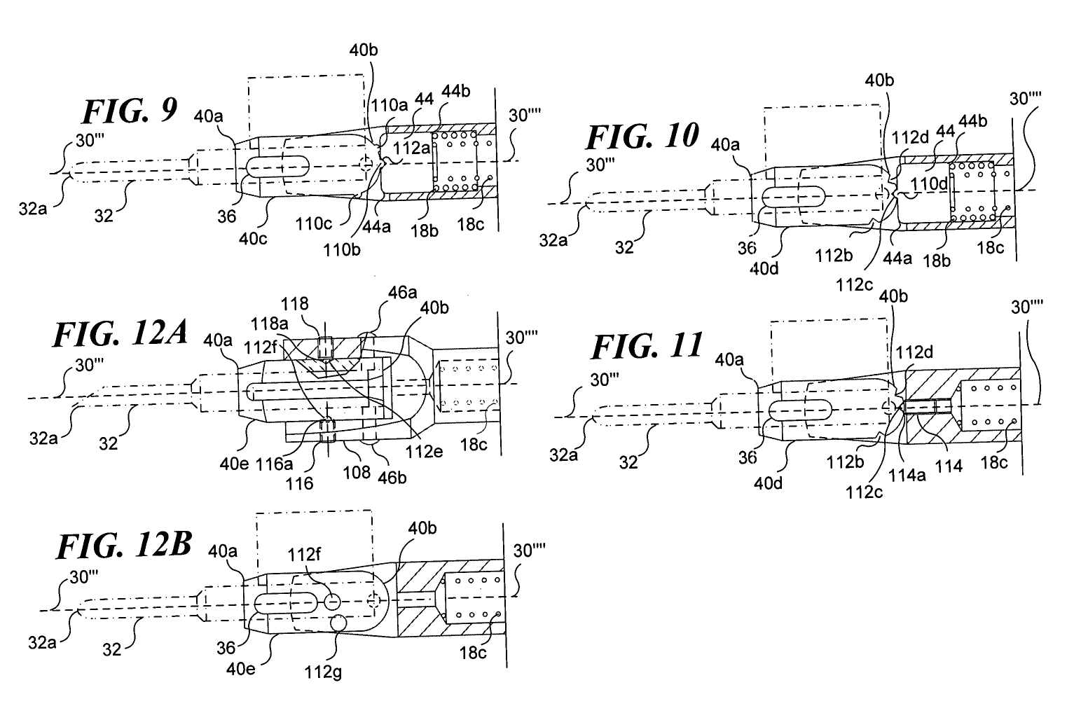

In an alternative indexing means, the pivotal portion of the housing may include a plurality of protrusions, such as protrusion 110c, protrusion 110b, and protrusion 110a as shown in a cartridge receiver portion 40c of FIG. 9 or may include a plurality of notches, such as notch 112b, notch 112c, and notch 112d, as shown in a cartridge receiver portion 40d of FIG. 10. The protrusion or notch interacts with sliding block 44 that includes respectively, either a notch 112a (FIG. 9), or a protrusion 110d (FIG. 10) disposed at distal end 44a. Similar to FIG. 5 and as described above, helical spring 18b and helical spring 18c together apply a biasing force against sliding block 44 that urges either the notch of the sliding block to contain one of the protrusions (FIG. 9), or the protrusion of the sliding block to be inserted into one of the notches (FIG. 10) with sufficient force to provide resistance against the protrusion inadvertently moving to a different protrusion, or the notch inadvertently moving to a different notch.

Although not shown, when the user wants to load the IOL cartridge into the cartridge receiver portion, the pivotal portion is pivoted so that protrusion 110c (FIG. 9) is inserted into notch 112a of the sliding block, or so that notch 112b (FIG. 10) contains protrusion 110d of the sliding block. When the user wants to pivot the cartridge receiver portion to a different angle, the pivotal portion is pivoted such that either protrusion 110b (FIG. 9), or protrusion 110a (FIG. 9) is respectively inserted into notch 112a (FIG. 9). Similarly, when the user wants to pivot the cartridge receiver portion to a different angle, the pivotal portion is pivoted such that either notch 112c (FIG. 10), or notch 112d (FIG. 10) contains protrusion 110d (FIG. 10). Helical spring 18b and helical spring 18c again provide the total biasing force acting on the sliding block to stabilize the cartridge receiver portion at an angle, as desired. Those skilled in the art will recognize that the number of protrusions or notches that may be formed on the proximal end of the pivotal portion can readily be increased or decreased in order to provide either more or fewer discrete positions at which the pivotal portion of the housing can be selectively set and retained.

Also, those skilled in the art will recognize that only one or more than two helical springs may be provided to produce the biasing force used to stabilize the cartridge receiver portion at each different desired angle relative to the handle portion of the housing. For example, in the configuration of FIG. 11, sliding block 44 has been replaced by a ball spring screw 114. An internal spring (not shown) of the ball spring screw applies a biasing force against a ball 114a disposed at the distal end of the ball spring screw that urges the ball into notch 112c with sufficient force to provide resistance against the notch inadvertently moving to a different notch. Although it is not shown, when the user wants to load the IOL cartridge into the cartridge receiver portion, the pivotal portion is pivoted so that ball 114a is inserted into notch 112b. When the user wants to pivot the cartridge receiver portion to a different angle, the user applies sufficient force such that ball 114a is forced from the notch and back into the ball spring screw. When the ball is aligned with notch 112c or notch 112d, it is biased into the notch, thereby stabilizing the cartridge receiver portion at the corresponding angle.

FIG. 12A and FIG. 12B illustrate cartridge receiver portion 40e that lacks the sliding block and which includes a ball spring screw 116 and a ball spring screw 118. Ball spring screw 116 is disposed on the elongate housing adjacent to the distal end of the handle portion, so that a ball 116a engages one side of cartridge receiver portion 40e, because ball 116a is biased inwardly towards longitudinal axis 30′″ and into an indentation 112f disposed on the outer wall of the cartridge receiver portion, between proximal end 40b and distal end 40a.

Ball spring screw 118 is similarly disposed on the elongate housing adjacent to the distal end of the handle portion, on an opposite side from ball spring screw 116, so a ball 118a engages the other side of cartridge receiver portion 40e, because ball 118a is also biased inwardly toward longitudinal axis 30′″ and into an indentation 112e disposed on the opposite outer wall of the cartridge receiver portion, between proximal end 40b and distal end 40a. The combination of forces exerted by ball 116a and ball 118a ensure that the pivotal portion does not inadvertently move to a different position.

However, when the user wants to load the IOL cartridge into the cartridge applies sufficient force such that ball 116a and ball 118a are forced out of engagement with their respective indentations. When ball 116a and ball 118a are aligned with a different indentation, for example, such as an indentation 112g (FIG. 12B), ball 116a and ball 118a are biased into the different indentations, thereby stabilizing the cartridge receiver portion at a different desired angle.

Those skilled in the art will realize that there are additional variations beyond the five configurations discussed above for practicing the indexing means, and thus, this disclosure is not limited to the five indexing means described above.

Furthermore, the inserter may be configured such that the cartridge receiver portion is fixedly mounted to the handle portion at a predefined angle, instead of being pivotally mounted to the handle portion. For example, angle 48b, as shown in FIG. 8C, may be predefined, i.e. fixed, such that the cartridge receiver portion does not pivot with respect to the handle portion and thus, does not require any indexing means. For example, the user may attach a threaded proximal end of the cartridge receiver portion to a threaded distal end of the handle portion such that joining of the two pieces provides only one fixed angle between the two portions of the housing. In the alternative, the handle portion and the cartridge receiver portion may be provided by the manufacturer already fixed at the predefined angle relative to each other, so that the user does not need to assemble the pieces prior to using the inserter.

Housing 12c also hosts a slide 50 which has a reduced diameter bore 50a (FIG. 5) that prevents excessive buckling of a flexible advancer used in an alternative mover 52b (FIG. 5) that is employed with housing 12c (FIG. 5), as discussed in greater detail below.

Those skilled in the art will recognize that the three housing configurations discussed above are not limited to configurations that are only adapted to accept IOL cartridges in cartridge receiver opening 28a (FIG. 2A) or 28b (FIG. 2B), with distal ends that extend through distal opening 26b (FIG. 1A or FIG. 1B). For example, the housing can be configured so that the cartridge receiver opening is formed as a slot that extends to the distal end of the housing, thus eliminating a distinct and separate distal opening in the housing, yet still securing the cartridge within the housing. In addition, with the distinct and separate distal opening in the housing eliminated, the IOL cartridge may also be loaded into the housing through distal end 14a. Subsequently, the IOL cartridge may be secured within the housing by various means, such as a bayonet lock, where the IOL cartridge is inserted and then rotated into a secured position.

### Mover Configurations

FIG. 4A illustrates one configuration of a mover, mover 52a that is configured to slide along longitudinal axis 30′ (FIG. 1A). Mover 52a comprises a push rod 54 (or a push rod 54′ as shown in FIG. 4D and FIG. 4E) with distal end 54a (or a distal end 54a′ as shown in FIG. 4D and FIG. 4E) joined to a plunger 56 with proximal end 56b. Plunger 56 also includes pin 58, a first cavity 74a for accommodating the multi-step detent configurations to be described below, and a surface area 60 is disposed at the proximal end 56b of the plunger. Push rod 54 (and push rod 54′ as shown in FIG. 4D and FIG. 4E) is sized smaller in diameter than plunger 56 so that when the user moves the push rod along the longitudinal axis 30′ (FIG. 1A), distal end 54a (or distal end 54a′ as shown in FIG. 4D and FIG. 4E) of the push rod passes through distal end 32b (FIG. 3A) of the IOL cartridge and makes direct contact with the IOL that has been placed within the IOL cartridge. The artificial IOL is thereby pushed from the IOL cartridge at a user-controlled rate. Surface area 60 is provided on the proximal end of the push rod to enable the user to use a thumb or other digit to apply a longitudinal manual force 62a, a longitudinal manual force 62b, or a longitudinal manual force 62c, as shown respectively in FIGS. 4A, 6A, and 7A, to advance the mover along the longitudinal axis of the housing. As noted above, pin 58 (FIG. 4A) engages groove 22 (FIG. 1A) to limit the range of travel of the mover between distal end 22a (FIG. 1A) and proximal end 22b (FIG. 1A) of the groove. Pin 58 (FIG. 4A) and groove 22 (FIG. 1A) also cooperate to prevent the rotation of the mover about longitudinal axis 30″ (FIG. 4A).

FIG. 5 illustrates mover 52b enclosed within housing 12c, which includes the cartridge receiver portion that is selectively pivotal. Instead of push rod 54, mover 52b includes advancer 64 (FIGS. 6C, 7C, 8C) having a distal end 64a (FIG. 6C) and a proximal end 64b (FIG. 5) that is joined to plunger 56. The advancer is sufficiently flexible (for example, it is preferably fabricated from a multi-strand stainless steel wire cable) so that it bends as advanced around the acute angle at which cartridge receiver portion 40 (FIG. 1C) is positioned relative to the handle portion of the housing. Alternatively, the advancer may comprise a nonmetallic material that is elastic. The advancer is preferably prevented from buckling, as described above, by providing slide 50 with reduced diameter bore 50a (FIG. 1C), within the handle portion. The reduced diameter bore minimizes any transverse deflection of the advancer relative to the longitudinal axis of the handle portion, which might otherwise occur because the advancer is flexible.

Distal end 64a (FIG. 6C) of the advancer is configured to pass through the cartridge receiver portion and controllably force an artificial IOL therefrom and into an eye. Surface area 60 (FIG. 6C) is again provided on the proximal end of the plunger to enable the user to apply longitudinal manual force 62b, or longitudinal manual force 62c as shown in FIG. 6C, and FIG. 7C to move mover 52b to various locations along the longitudinal axis. Likewise, pin 58 (FIG. 6C) is also disposed such it protrudes from the plunger and engages groove 22 to limit the range of travel of the mover and inhibit it from rotating relative to the housing.

### Multi-Step Detent Configurations and Dispositions

FIG. 4A shows a first configuration of a multi-step detent, a cantilevered multi-step detent extending from plunger 56 of the mover. Cantilevered multi-step detent 66a includes a longitudinally extending component 68 having a distal end 68a and a proximal end 68b, a tab 70, an outer edge 72, a stair step 76a with a distal face 78a, and a stair step 76b that extends radially further outwardly from the mover than stair step 76a and has a distal face 78b. The longitudinally extending component is mounted to plunger 56 by a pin 80 that supports distal end 68a, so that the longitudinally extending component is cantilevered from pin 80 and so that the stair steps project outwardly from first cavity 74a that is formed in the proximal end of the plunger.

The multi-step detent is disposed towards the proximal end of the plunger in which a second orifice 86 (FIG. 6A) is disposed. Longitudinally directed force applied to proximal end 56b of the plunger advances the mover (i.e., either mover 52a or mover 52b) along the longitudinal axis of the housings, in successive predefined intervals, so that the motion is limited and stops specifically at a first detent position, a second detent position and a completely advanced position. In this manner, an artificial IOL is controllably expelled into an eye by applying the manual force to surface area 60, which is disposed at the proximal end 56b of the plunger.

The first detent position is defined at the point where the mover has been advanced distally along longitudinal axis 30′, for example, by a surgical assistant or nurse, until distal face 78a abuts housing 12a, as shown in FIG. 6A. At this point, the artificial IOL will have been advanced into the distal portion of the IOL cartridge, and is thus prepped for the ocular surgeon to insert the IOL into the eye. Subsequently, outer edge 72 (FIG. 6A) is depressed below the housing, enabling the mover to be further advanced by the ocular surgeon to the second detent position, where face 78b abuts housing 12a, as shown in FIG. 7A. At this point, the artificial IOL will normally have been advanced so that part of it extends from the distal end 32a of the IOL cartridge and within the eye.

If the user attempts to advance the mover by over depressing the multi-step detent into first cavity 74a (FIG. 4A) so that distal face 78a (FIG. 4A) does not abut housing 12a (FIG. 1A) and fail to stop the advancement at the first detent position, tab 70 (FIG. 4A) will be forced outwardly through second orifice 86 (FIG. 6A) and abut the housing 12a, preventing the mover from being advanced any further. The tab is thus an optional safety feature that will help to prevent an uncontrolled ejection of the artificial IOL from the IOL cartridge and into a patient's eye. Tab 70 is disposed in the second opening along an edge of longitudinally extending component 68 (FIG. 4A) that is opposite outer edge 72 (FIG. 4A) on which the plurality of stair steps are disposed. In order to deactivate the tab, the user simply releases pressure on outer edge 72 (FIG. 4A) so that the longitudinally extending component is biased outwardly again in its original position and thus, tab 70 (FIG. 4A) is enclosed within the plunger and no longer abuts against the housing as an impediment to further advancement of the mover. The surgeon can again depress longitudinally extending component 68 (FIG. 4A) and continue advancing the plunger as necessary to fully deploy the IOL within the eye, and the plunger can optionally be fully advanced, as shown in FIG. 8A, e.g., as the inserter is withdrawn from the eye.

FIG. 4B shows a spring-loaded multi-step detent 66b that is similar to cantilevered multi-step detent 66a, as described above. However, spring-loaded multi-step detent 66b includes a helical spring 82 that biases the longitudinal extending component outwardly instead of cantilever 80 (FIG. 4A). Distal end 68a (FIG. 4B) of the longitudinal extending component rotates about a pivot 84. Helical spring 82 is disposed beneath outer edge 72, and inside first cavity 74a. When a user depresses outer edge 72 inwardly, helical spring 82 is compressed and provides a resisting force.

A distal end 54a of mover 52a is inserted into the proximal opening 26a of either housing 12a (FIG. 1A) or housing 12b (FIG. 1B) so that longitudinal axis 30″ is aligned with longitudinal axis 30′. Thus, the mover travels distally along longitudinal axis 30′ of the internal bore of either of these housings between successive detent steps, as described above in regard to cantilevered multi-step detent 66a (FIG. 4A) as longitudinal force is applied to the mover.

FIG. 4D shows a second configuration of a multi-step detent, cantilevered multi-step detent 66f that includes longitudinally extending component 68. Longitudinally extending component 68 has distal end 68a, outer edge 72, and a protrusion 120 with an angled distal face 120a and an angled proximal face 120b that extends radially outwardly from the mover. Angled distal faces 120a and 120b form substantially obtuse angles with respect to outer edge 72. Similar to multi-step detent 66a, longitudinally extending component 68 is mounted to plunger 56 by pin 80 that supports distal end 68a, so that the longitudinally extending component is cantilevered from pin 80 and so that the protrusion projects outwardly from first cavity 74a that is formed in the proximal end of the plunger.

Also in a manner similar to the multi-step detents described above, this multi-step detent is disposed towards the proximal end of the plunger. A longitudinal manual force applied to proximal end 56b of the plunger advances the mover (i.e., either mover 52a or mover 52b) along the longitudinal axis of the housing, in successive predefined intervals, so that the motion is limited and so that the mover stops specifically at a first detent position, a second detent position, and at a completely advanced position.

However, unlike the other configurations of the multi-step detents, this configuration only requires one hand to advance the mover to its various detent positions. The first detent position is defined at the point where the mover has been advanced distally along longitudinal axis 30′ sufficiently by longitudinal manual force 62a until the base of angled distal face 120a abuts housing 12b, as shown in FIG. 6D. Subsequently, protrusion 120 is gradually and smoothly recessed into the plunger cavity as sufficient longitudinal manual force 62b is applied for example, by the surgeon's thumb, to bias the protrusion into the plunger cavity where it slides along the inner surface of the housing until the projection can fit into proximal orifice 96e (FIG. 1D), as shown in FIG. 7D, at a second detent position. Thus, angled distal face 120a provides enough resistance when it abuts the edge of the housing to indicate to the surgeon that a particular detent position has been reached, but because of its distal angled surface, it can subsequently be forced to smoothly slide into the plunger cavity as the surgeon applies sufficient longitudinal force to overcome the resistance when the projection abuts the housing. Thus, when advancing the plunger from the second detent position, the surgeon again applies sufficient longitudinal manual force 62c to disengage protrusion 120 from proximal orifice 96e, enabling the plunger to be advanced as necessary to fully deploy the IOL within the eye. The plunger can thus optionally be fully advanced, as shown in FIG. 8D.

FIG. 4E shows the second configuration of a cantilevered multi-step detent 66g with a disengager that includes longitudinally extending component 68 having distal end 68a, outer edge 72, and protrusion 124. The protrusion again has angled distal face 124a and proximal face 124b and extends radially outwardly from the mover. Angled distal face 124a forms substantially an obtuse angle with respect to outer edge 72 so that multi-step detent 66g works in a manner similar to cantilevered multi-step detent 66f Proximal face 124b forms substantially a right angle with respect to outer edge 72. Multi-step detent 66g also includes a disengager 122 disposed proximal of protrusion 124, and having an angled distal face 122a.

However, because a surgeon is controllably advancing the mover, if the surgeon advances the mover slowly enough, there may not be an audible sound when protrusion 120 (FIG. 4D) of multi-step detent 66f is biased outward and engages proximal orifice 96e (FIG. 1D), as would be the case when protrusion 124 (FIG. 4E) is biased outwardly into proximal orifice 96e (FIG. 1D). In that case, an audible clicking sound is produced as outer edge 72 that is inside the housing comes into contact with the internal surface area of the housing as the protrusion slips into and engages the proximal orifice. Thus, since the surgeon may prefer to use multi-step detent 66g because of its more audible click indicating that the second detent position has been reached, it is necessary that disengager 122 be included in this configuration, to enable the mover to be advanced proximally to its original position after use by releasing the protrusion from the proximal orifice. Specifically, when the surgeon has advanced the multi-step detent beyond its second detent position such that protrusion 124 is forcibly disengaged from the proximal orifice and the surgeon releases longitudinal manual force 62c, housing spring 18a will cause the advancer to travel proximally. Eventually as a result of the biasing force provided by housing spring 18a and/or as a result of the surgeon applying a longitudinal manual force (not shown) to retract the plunger proximally, protrusion 124 will once again be biased outwardly into engagement with proximal orifice 96e. However, the surgeon will not be able to advance the mover to its original position, as shown in FIG. 4E, by simply pulling on the mover, because proximal face 124b forms a right angle with respect to the outer surface and does not automatically disengage the protrusion from the proximal orifice when the plunger is retracted proximally. However, the surgeon can depress disengager 122 to disengage protrusion 124 from the proximal orifice and and enable the plunger to be withdrawn proximally until it is in the position shown in FIG. 4E.

### Manipulation of IOL Cartridge into Intraocular Lens Inserter

Typically, a surgical assistant or nurse will place the artificial IOL into IOL cartridge 32 (FIG. 3A) or IOL cartridge 34 (FIG. 3B) and load the IOL cartridge and IOL into the housing of the intraocular lens inserter. In order to properly load the IOL cartridge as shown in FIG. 3A into housing 12a, the surgical assistant will insert distal end 32a of IOL cartridge 32 into cartridge receiver opening 28a (FIG. 2A), such that distal end 32a (FIG. 3A) protrudes beyond distal opening 26b. Longitudinal axis 30′″ of the IOL cartridge is then aligned with longitudinal axis 30′ of the housing 12a. This alignment ensures that distal end 54a (FIG. 4A) of push rod 54 or distal end 54a′ (FIG. 4D or FIG. 4E) will make contact with the artificial IOL disposed within IOL cartridge 32 as the push rod is advanced along longitudinal axis 30′.

Similarly, the surgical assistant may insert distal end 34a (FIG. 3B) of IOL cartridge 34 into cartridge receiver opening 28b (FIG. 2B or FIG. 2D) of housing 12b after the IOL has been placed in the IOL cartridge, so that distal end 34a (FIG. 3B) protrudes beyond distal opening 26b (FIG. 2B or FIG. 2D). Longitudinal axis 30′″ of the IOL cartridge will also be similarly aligned with longitudinal axis 30′ of the housing 12b.

The IOL cartridge loading procedure, however, will vary if housing 12c that includes cartridge receiver portion 40 is used. The surgical assistant will pivot the cartridge receiver portion 40 from the position shown in FIG. 1C, until it forms approximately a 90° angle relative to the longitudinal axis of the handle portion of the housing, as shown in FIG. 5. Angled flat surface 42c abuts against distal end 44a of sliding block 44 to temporarily maintain this angle. The surgical assistant can then readily insert distal end of the IOL cartridge into the cartridge receiver portion and pivot the cartridge receiver portion and cartridge to a relatively straight position for example, as shown in FIG. 6C. Angled flat surface 42b abutting against distal end 44a of sliding block 44 will provide the stability required to keep the pivotal portion of the housing and the IOL cartridge at the desired angle.

### Manipulation of Device to a First Detent Position

Once the IOL cartridge is properly loaded into housing 12a, housing 12b, or housing 12c, as described above, the surgical assistant will apply a longitudinal manual force to surface area 60 with a thumb or other digit, so that mover 52a (or mover 52b) is advanced along the longitudinal axis of the housing until distal face 78a abuts against proximal opening 26a of the housing as shown in FIG. 6A, FIG. 6B, or FIG. 6C, or until angled distal face 120a or angled distal face 124a abuts against proximal opening 26a of the housing as shown in FIG. 6D and FIG. 6E, respectively. The mover has then been advanced to the first detent position. Although FIG. 6A, FIG. 6B, FIG. 6D, and FIG. 6E do not show the mover inside the housing, an aligning guide 88 (FIG. 4A, FIG. 4B, FIG. 4D, FIG. 4E, and FIG. 5) provides a visual reference indicating the location of the proximal opening of the housing. The aligning guide thus provides an indication of the disposition of proximal opening 26a of housing 12a where it abuts distal face 78a. Similarly, the aligning guide indicates the location of proximal opening 26a of housing 12b abutted by either distal face 78a, angled distal face 120a, or angled distal face 124a. Thus, in the case of mover 52a (FIG. 4A) as the manual force is applied, distal end 54a (FIG. 4A) of push rod 54 (FIG. 4A) or distal end 54a′ (FIG. 4D or FIG. 4E) of push rod 54′ (FIG. 4D or FIG. 4E) which is smaller in diameter than the plunger, enters into the IOL cartridge disposed within the cartridge receiver opening and makes contact with the artificial IOL. However, mover 52a (FIG. 6A) will not yet have started to compress optional helical spring 18a when the mover is positioned in the first detent position.

Similarly, the longitudinal manual force (not shown) is initially applied to mover 52b (FIG. 5), but only after the cartridge receiver portion has been rotated back to a relatively straight position. Then, distal end 64a (FIG. 6C) of the advancer, which is smaller in diameter than the plunger, enters into the IOL cartridge disposed within the cartridge receiver opening and makes contact with the artificial IOL. The artificial IOL is then advanced into a distal portion of the cartridge. The artificial IOL remains inside the distal portion of the cartridge, ensuring that the artificial IOL is disposed so that the ocular surgeon can then control advancement of the IOL into the eye from the IOL cartridge. In other words, the present invention ensures that the surgical assistant does not overshoot the proper position of the artificial IOL in the IOL cartridge before the surgeon begins to use the inserter. As indicated in FIG. 5 pin 58 is disposed proximate to the proximal end 22b, but when the mover has been disposed in a first detent position as shown in FIG. 6C, the pin has traveled approximately half way down the length of groove 22. However, mover 52b has not yet started to compress helical spring 18c when the mover is positioned in the first detent position.

Furthermore, the surgical assistant does not depress the outer edge 72 (FIG. 5) of the multi-step detent in order to advance the mover to the first detent position. If the surgical assistant should inadvertently depress the multi-step detent into the first cavity, the surgical assistant will become aware of this error, since tab 70 (FIG. 5) will protrude through second orifice 86 and abut against the housing, effectively preventing further advancement of the mover, until the surgical assistant releases the force on the multi-step detent.

### Manipulation of Device to a Second Detent Position Using Housing 12a or Housing 12b

Typically, at this point the surgeon will insert the distal end of the intraocular lens inserter into the patient's eye through a small incision that has previously been made, and will begin to advance the mover to controllably eject the artificial IOL into the eye from the IOL cartridge. In order to move the mover from the first detent position to the second detent position, using either housing 12a or housing 12b, and either multi-step detent 66a or multi-step detent 66b, the surgeon depresses outer edge 72 (FIG. 6A) and applies longitudinal manual force 62b (FIG. 6A) to surface area 60 and controllably advances the mover until face 78b abuts proximate opening 26a (FIG. 1A) of the housing, thereby reaching the second detent position, as shown in FIG. 7A. As the mover is advanced further through the internal bore, distal end 54a (FIG. 7A) of push rod 54 (FIG. 7A) further advances the artificial IOL along the longitudinal axis of the IOL cartridge so that a portion of the artificial IOL is exposed outside of distal end 32a (FIG. 3A) of the IOL cartridge. Thus, the second, successive predefined detent position of the multi-step detent prevents overshooting the moment when the artificial IOL starts to unfurl uncontrollably within the eye. The surgeon is able to controllably eject the artificial IOL into the eye at the desired rate, resulting in a safer procedure by avoiding the uncontrolled unfurling of the IOL.

The second detent position is illustrated by FIG. 7A and FIG. 7B. Again, aligning guide 88 visually provides a reference indicating the location of the proximal opening 26a (FIG. 1B) of the housing along the longitudinal axis when abutting face 78b.

In the alternative, using either housing 12b with the proximal orifice or housing 12a with the proximal orifice and multi-step detent 66f or multi-step detent 66g, the surgeon applies longitudinal manual force 62b (FIG. 6D or FIG. 6E) to surface area 60 and controllably advances the mover until protrusion 120 or protrusion 124 is biased into the plunger cavity and subsequently snaps into proximal orifice 96e (FIG. 1D), thereby reaching the second detent position, as shown in FIG. 7D or FIG. 7E. Again, aligning guide 88 visually provides a reference indicating the location of proximal orifice 96e (FIG. 1D) along the longitudinal axis when protrusion 120 (FIG. 7D) or protrusion 124 (FIG. 7E) is biased outwardly into the proximal orifice.

### Manipulation of Device to a Second Detent Position Using Housing 12c

Before advancing the mover from the first detent position to the second detent position using housing 12c, the surgeon (or surgical assistant) will pivot the cartridge receiver portion 40 relative to the handle portion, for example, to a desired angle, such as angle 48b, as shown in FIG. 7C, which typically will be an angle of between twenty to thirty degrees. Angled flat surface 42a then abuts against distal end 44a of sliding block 44. The surgeon will similarly depress outer edge 72 (FIG. 6C) and apply longitudinal manual force 62b (FIG. 6C) to surface area 60 (FIG. 6C) to controllably advance the mover until distal face 78b abuts housing 12c at the second detent position, as shown in FIG. 7C. The mover is advanced further through the internal bore, and distal end 64a (FIG. 7C) of advancer 64 (FIG. 7C) in turn further advances the artificial IOL along the longitudinal axis of the IOL cartridge. Advancer 64 is sufficiently flexible enough so that the advancer bends when moving through angle 48b and does not buckle inside the housing because of reduced diameter bore 50a (FIG. 5). Pin 58 has traveled further along groove 22, as shown in FIG. 7C, and helical spring 18c is more compressed than as shown in FIG. 6C.

Also, helical spring 18c disposed in the intermediate portion of the housing will begin to provide a biasing force that resists advancing the mover through the internal bore along the longitudinal axis, as will be evident from the compression of the helical spring shown in FIG. 6C, compared to its state in FIG. 7C. In addition, helical spring 18b pushes against proximal end 44b (FIG. 7C) of sliding block 44 (FIG. 7C) and provides most of the force that biases the distal end 44a of sliding block 44 against angled fiat surface 42a in order to stabilize the cartridge receiver portion at angle 48b (FIG. 7C).

### Manipulation to a Fully Extended Position

The surgeon may ensure the full deployment of the IOL by advancing the push rod or advancer even further, up to the fully extended position, by depressing outer edge 72 (FIG. 7A) and applying longitudinal manual force 62c (FIG. 7A) to advance the mover after face 78b no longer abuts the proximal opening 26a (FIG. 7A, 7B, or 7C) of the housing. This advancement can continue until surface area 60 abuts proximal opening 26a of the housing, as shown in FIG. 8A, FIG. 8B, and FIG. 8C. Once again, this position is apparent from alignment guide 88 in FIG. 8A and FIG. 8B, and apparent from FIG. 8C. Note that in the fully advanced position, pin 58 has traveled the length of groove 22 (FIG. 1A, FIG. 1B, and FIG. 8C) and rests against distal end 22a (FIG. 1A, FIG. 1B, and FIG. 8C) of the groove. Also, helical spring 18c is fully compressed, as shown in FIG. 8C.

In the alternative, the surgeon may ensure the full deployment of the IOL by advancing the push rod or advancer even further, up to the fully extended position, by applying longitudinal manual force 62c (FIG. 7D or FIG. 7E) to advance the mover such that protrusion 120 or protrusion 124 is forcibly disengaged from proximal orifice 96e and thus slides into the plunger cavity, in a controlled advancement of the push rod. Note that if the surgeon is utilizing multi-step detent 66g, angled distal face 122a will provide additional resistance as it meets housing opening 26a prior to disengager 122 being smoothly forced into the plunger cavity as the push rod is advanced. This additional resistance ensures further control of the push rod or advancer. This advancement can continue until surface area 60 abuts proximal opening 26a of the housing, as shown in FIG. 8D or FIG. 8E. Note that disengager 122 is sized such that it cannot fit into proximal orifice 96e. Once again, this position is apparent from alignment guide 88 in FIG. 8D and FIG. 8E.

If optional helical spring 18a (FIG. 1A, FIG. 1B, and FIG. 1D) is disposed in the intermediate portion of the housing, the helical spring will provide a force that resists advancing the mover through the internal bore along the longitudinal axis. The force of this spring will further ensure that the lens is controllably ejected, since the resisting force will impede the surgeon's inadvertent excessive manual force to facilitate an even slower controlled advancement of the mover along the longitudinal axis through the internal bore.

As the IOL is fully deployed in the eye, the surgeon will begin to withdraw distal end 54a or distal end 54a′ (FIG. 7A, FIG. 7D, or FIG. 7E) of the push rod or the distal end 64a (FIG. 7C) of the advancer from the eye. Thus, when the surgeon stops applying longitudinal manual force 62c, helical spring 18c will then provide the biasing force that moves the advancer or the push rod proximally along the longitudinal axis until it is withdrawn to an intermediate position within the housing. However, if the inserter being used includes multi-step detent 66g, protrusion 124 is biased outwardly into the proximal orifice and prevented from traveling proximally since proximal face 124b abuts the proximal portion of the housing orifice. However, the user can then depress disengager 122, thereby forcing protrusion 124 from the proximal orifice and into the plunger cavity, so that proximal advancement can continue.

During the IOL insertion procedure, annular ring 24 (FIG. 1A, 1B, 1D, or 8C) is used in combination with surface area 60 to enable the surgeon to grasp the intraocular lens inserter and more efficiently apply the longitudinally directed manual force. Preferably, annular ring 24 readily rotates about the housing.

### Additional Configurations of the Multi-Step Detent

An alternative fifth preferred embodiment of the inserter includes a third configuration of the multi-step detent, multi-step detent 66c, as shown in FIG. 13A, FIG. 13B, and FIG. 13C. FIG. 13A illustrates multi-step detent 66c, which includes a plunger spring 92a, a plunger spring 92b, a pin 94a, a pin 94b, a detent releaser arm 98a, and a detent releaser 100a. Housing 12c (it will be understood that the multi-step detents discussed below can optionally be included in any configuration of the housing discussed above) includes a housing orifice 96a that is sized to accommodate pin 94a and pin 94b. The detent releaser arm extends over housing orifice 96a. A mover 52c is configured to include a second cavity 90a in addition to first cavity 74a. Pin 94a is biased outwardly by plunger spring 92a and is contained within first cavity 74a. Pin 94b is similarly biased outward by plunger spring 92b and is contained within second cavity 90a. The detent releaser is disposed adjacent to housing orifice 96a. FIG. 13A illustrates only a portion of intraocular lens inserter 10 (FIG. 8C) prior to engagement of the multi-step detent in the first detent position. The relative distance shown between the first cavity and the second cavity are not to scale.

When the surgeon applies longitudinal manual force 62a to controllably advance mover 52c along longitudinal axis 30′, pin 94a, which is biased by plunger spring 92a will move into housing orifice 96a, (FIG. 13A) so that movement of the mover along the longitudinal axis will be stopped at this first detent position, as shown in FIG. 13B.

In order to advance the mover from the first detent position to the second detent position, the surgeon will apply manual force 62d (FIG. 13B) to depress detent releaser 100a, which is disposed under detent releaser arm 98a, into housing orifice 96a (FIG. 13B). The detent releaser will depress pin 94a back into the first cavity, releasing the mover to be further advanced. While maintaining manual force 62d, the surgeon will apply longitudinal manual force 62b to advance mover 52c further along the longitudinal axis until pin 94b that is loaded by plunger spring 92b is biased into housing orifice 96a (FIG. 13C). In this second detent position, any further advancement of the mover 52c along the longitudinal axis 30′ is prevented, as shown in FIG. 13C.

If the surgeon decides to advance the mover further along the longitudinal axis 30′, the surgeon may apply manual force 62e such that detent releaser 100a is inserted into housing orifice 96a thereby forcing pin 94b out of the orifice and enabling the plunger to be further controllably advanced along the longitudinal axis by applying longitudinal manual force 62c, at least until surface area 60 (not shown) abuts the proximal opening of housing 12c.

A sixth embodiment of an inserter, shows a fourth configuration of a multi-step detent, multi-step detent 66d, which is disposed in a first cavity 74b, as illustrated in FIGS. 14A, 14B, and 14C. With reference to FIG. 14A, multi-step detent 66d includes first detent releaser 100a, and a second detent releaser 100b. These releasers are disposed under an arm 98b that is mounted to the outer surface of the elongate housing. This elongate housing includes a first orifice 96b and a second orifice 96c that are spaced apart along longitudinal axis 30′ at positions defining the first detent position and the second detent position. The first detent releaser is adjacent to the first orifice, and the second detent releaser is adjacent to the second orifice. A mover 52d also includes first cavity 74b and a pin 94c. Pin 94c is disposed within the first cavity and is biased outwardly by a cantilevered spring arm 102. The pin is sized to fit within the first orifice and the second orifice. FIGS. 14A, 14B, and 14C only illustrate a portion of intraocular lens inserter 10 (FIG. 8C). FIG. 14A shows the disposition of the multi-step detent and mover prior to engagement of the multi-step detent in the first detent position.

When the surgeon applies longitudinal manual force 62a (FIG. 14A) to controllably advance mover 52d along longitudinal axis 30′, pin 94c is biased by cantilevered spring arm 102 into first orifice 96b so that movement of the mover along the longitudinal axis is impeded in the first detent position, as shown in FIG. 14B.

In order to advance the mover from the first detent position to the second detent position, it is necessary for the surgeon to apply inwardly directed manual force 62f such that detent releaser 100a, which is disposed under detent releaser arm 98b, is inserted into first orifice 96b (FIG. 14B), thus forcing pin 94c back into the first cavity and enabling further advancement of the mover. As shown in FIG. 14B, while maintaining manual force 62f, the surgeon will apply longitudinal manual force 62b to controllably advance mover 52d further along the longitudinal axis, until pin 94c is aligned with and biased into second orifice 96c by cantilevered spring arm 102. Thus, further advancement of the mover 52d along the longitudinal axis 30′ from this second detent position is prevented, as shown in FIG. 14C.

When the surgeon decides to advance the mover further along the longitudinal axis 30′, the surgeon applies manual force 62g, as shown in FIG. 14C, so that detent releaser 100b is inserted into second orifice 96c, forcing pin 94c from the orifice and enabling the mover to be further controllably advanced along the longitudinal axis by applying longitudinal manual force 62c, at least until surface area 60 (not shown) abuts the proximal opening of housing 12c.

The seventh embodiment of an inserter includes a fifth configuration of a multi-step detent, multi-step detent 66e, as shown in FIGS. 15A, 15B, and 15C. Multi-step detent 66e includes a collar 104 that is disposed adjacent to an orifice 96d formed in the housing. Collar 104 includes a pin 94d, and the collar is biased to force the pin inwardly through the orifice by a helical-type collar spring 106. Collar spring 106 is disposed between the exterior surface of the elongate housing and the radially inner surface of the collar, at a point opposite pin 94d. A mover 52e also includes first cavity 74a and a second cavity 90c, which are each sized to receive pin 94d, when it passes through orifice 96d. FIG. 15A illustrates a portion of intraocular lens inserter 10 prior to engagement of multi-step detent 66e in the first detent position. In this Figure, collar spring 106 is shown in a compressed state such that pin 94d is biased inwardly, pushing on mover 52e.

When the surgeon applies longitudinal manual force 62a, as shown in FIG. 15A, and controllably advances mover 52e along longitudinal axis 30′, pin 94d moves into first cavity 74a as the first cavity becomes aligned with orifice 96d. Engagement of pin 94d into first cavity 74a occurs at the first detent position, such that movement of the mover along the longitudinal axis is impeded as shown in FIG. 15B.

In order to advance the mover from the first detent position to the second detent position, the surgeon applies a manual force 62h, as shown in FIG. 15B, such that collar spring 106 is compressed by the collar and pin 94d disengages from first cavity 74a. Pin 94d is then allowed to be reset in a biased position as shown in FIG. 15A, but only after the user has advanced mover 52e along the longitudinal axis by applying longitudinal manual force 62b. When second cavity 90c is aligned with orifice 96d, pin 94d will be forced into engagement with cavity 90c, so that movement of the mover along the longitudinal axis is impeded at the second detent position, as shown in FIG. 15C.

When the surgeon wants to controllably advance the mover further along longitudinal axis 30′, the surgeon again applies a manual force 62i as shown in FIG. 15C, so that collar spring 106 is compressed, and pin 94d is forced from cavity 90c (FIG. 15A). Longitudinal manual force 62c is applied to advance the mover along the longitudinal axis, as desired, limited by surface area 60 (not shown) abutting against the proximal opening of housing 12c.

Although the present invention has been described in connection with the preferred form of practicing it and modifications thereto, those of ordinary skill in the art will understand that many other modifications can be made to the present invention within the scope of the claims that follow. Accordingly, it is not intended that the scope of the invention in any way be limited by the above description, but instead be determined entirely by reference to the claims that follow.

1. An intraocular lens inserter for controllably expelling an artificial lens into an eye in at least one defined increment, comprising:
(a) a housing that constrains advancement of an artificial lens along a longitudinal path within the housing, the housing confining a rod to travel along the longitudinal path as an operator pushes on an end of the rod to advance an artificial lens into an eye; and
(b) means for limiting the advancement of the rod along the longitudinal path to the at least one defined increment, said means being operative when activated by an operator, to allow the rod to advance through a next increment, for thus controllably expelling an artificial lens into an eye.

2. An intraocular lens inserter for implanting an artificial lens into an eye in a controlled manner, comprising:
(a) an elongate housing having an internal bore, with a distal end and a proximal end, and having a plurality of openings, including:
(i) a cartridge receiver opening disposed proximate the distal end of the internal bore, said cartridge receiver opening being adapted to receive a cartridge in which an artificial lens is disposed and which is formed to enable an artificial lens to be forced outwardly therefrom and into an eye; and
(ii) an opening disposed at the proximal end of the internal bore;
(b) a mover sized to slide along a longitudinal axis of the internal bore within the elongate housing and having a distal end that is adapted to pass through a cartridge and force an intraocular lens therefrom and into an eye, and a proximal end adapted to receive a manual force applied by an operator to advance the mover along the longitudinal axis of the internal bore; and
(c) a detent that interacts with the elongate housing to controllably limit an advancement of the mover through the internal bore to at least one predefined interval, the detent being biased to engage the elongate housing at the at least one predefined interval, further advancement of the mover being limited by the detent until the detent is forcibly released from engaging the housing to enable the mover to be advanced by a user, enabling an intraocular lens to be controllably expelled from a cartridge and into an eye by the distal end of the mover as it advances distally through a cartridge.

3. The intraocular lens inserter of claim 2, wherein the mover includes a push rod joined to a plunger, the push rod being disposed adjacent to the distal end of the internal bore, and the plunger being disposed adjacent to the proximal end of the internal bore, the push rod being substantially smaller in a cross-sectional size than the plunger.

4. The intraocular lens inserter of claim 3, further comprising a pin disposed in the plunger that extends into a groove formed on an inner wall of the elongate housing, so that the pin and the groove cooperate to prevent the mover from rotating around the longitudinal axis of the internal bore.

5. The intraocular lens inserter of claim 4, further comprising an insert retained inside of the housing, said insert having an inner diameter that defines a cross-sectional size of the internal bore, so that the mover is slidable longitudinally along the internal bore, and a groove formed therein that receives a pin disposed on the plunger, the groove being formed within the insert and extending along a portion of its longitudinal extent, so that an engagement of the pin by the groove prevents the plunger from being rotated within the internal bore.

6. The intraocular lens inserter of claim 3, wherein the at least one predefined interval controlled by the detent includes one of:
(a) a first detent position that limits the push rod advancement to a first point where an intraocular lens has been advanced into a distal portion of a cartridge fitted into the intraocular lens inserter; and
(b) a second detent position that limits the push rod advancement to a second point where an intraocular lens is advanced sufficiently so that a portion of an intraocular lens extends from the distal end of a cartridge.

7. The intraocular lens inserter of claim 3, further comprising a spring that is disposed in the internal bore, between an intermediate point in the housing and a distal end of the plunger to provide a force that resists advancing the mover distally through the internal bore over at least a portion of its travel, when controllably expelling an intraocular lens from a cartridge with force applied by an operator to move the plunger.

8. The intraocular lens inserter of claim 6, further comprising a first cavity formed in the plunger for accommodating the detent.

9. The intraocular lens inserter of claim 8, wherein the detent comprises a longitudinally extending component having an outer edge, a distal end, and a proximal end, said longitudinally extending component having at least one stair step formed along the outer edge, each of the at least one stair step extending outwardly from the first cavity to a different level, a face on each of the at least one stair step that is transverse to the longitudinal axis of the internal bore abutting against a portion of the elongate housing to engage the elongate housing at the at least one predefined interval, until the detent is depressed into the first cavity to clear its abutment against the elongate housing.

10. The intraocular lens inserter of claim 9, wherein the distal end of the longitudinally extending component is mounted to the plunger by a cantilever, so that the at least one stair step is biased outwardly from the first cavity by the cantilever.

11. The intraocular lens inserter of claim 9, further comprising a spring disposed under the proximal end of the longitudinally extending component, the spring biasing the at least one stair step outwardly from the first cavity.

12. The intraocular lens inserter of claim 8, further comprising a second cavity formed in the plunger, wherein the detent further comprises a tab formed along an edge that is opposite the edge on which the plurality of stair steps are formed, the tab extending outwardly from the second cavity and abutting against a portion of the elongate housing to engage the elongate housing prior to the at least one predefined interval, if the plurality of stair steps are depressed excessively, said tab preventing an uncontrolled advancement of the push rod by preventing the plurality of stair steps from being depressed into the first cavity by an excessive amount, so that the at least one predefined interval of controlled advancement is avoided.

13. The intraocular lens inserter of claim 8, further comprising a second cavity formed in the plunger, said detent comprising a first pin disposed in the first cavity and biased outwardly therefrom, and a second pin disposed in the second cavity and biased outwardly therefrom.

14. The intraocular lens inserter of claim 13, further comprising an orifice formed in the elongate housing and sized to accommodate one of the first pin and the second pin, the first pin being biased to move into the orifice to limit the advancement of the mover through the internal bore at one interval, and the second pin being biased to move into the orifice to limit the advancement of the mover through the internal bore at a successive interval, wherein the detent further comprises an arm mounted to the elongate housing and having a detent releaser that is disposed adjacent to the orifice in the elongate housing, said detent releaser being operative when depressed into the orifice to move one of the first pin and the second pin out of engagement with the orifice, at successive intervals of advancement of the mover through the internal bore of the elongate housing.

15. The intraocular lens inserter of claim 14, wherein the detent further comprises an arm mounted to the elongate housing, extending over the orifice and having a detent releaser that is disposed adjacent to the orifice in the elongate housing to move into the orifice when a radially inward directed force is applied to the arm, said detent releaser being operative when depressed into the orifice to move the first pin and then the second pin out of engagement with the orifice, at successive intervals of advancement of the mover through the elongate housing.

16. The intraocular lens inserter of claim 8, further comprising a first orifice, and a second orifice longitudinally spaced-apart from the first orifice, the first orifice and the second orifice being formed in the elongate housing, said detent being disposed in the first cavity and comprising a cantilevered spring arm having a pin disposed on an outer surface of the cantilevered spring arm and sized to fit into engagement within the first orifice and the second orifice at successive intervals of advancement of the mover through the internal bore of the elongate housing.

17. The intraocular lens inserter of claim 16, wherein the detent further comprises an arm mounted to the elongate housing, extending over the first orifice and the second orifice, and having a first detent releaser and a second detent releaser that are disposed on an undersurface of the arm, adjacent to the first orifice and the second orifice, respectively, said first detent releaser being depressed into the first orifice to move the pin out of engagement with the first orifice, and said second detent releaser being depressed into the second orifice to move the pin out of engagement with the second orifice, at successive intervals of advancement of the mover through the internal bore of the elongate housing.

18. The intraocular lens inserter of claim 8, further comprising a second cavity formed in the plunger, and wherein the elongate housing includes an orifice.

19. The intraocular lens inserter of claim 18, wherein the detent comprises a collar around the elongate housing, adjacent to the orifice, said collar having a pin disposed on a radially inner surface thereof, adjacent to the orifice and sized to fit within the orifice, and a spring disposed between the exterior of the elongate housing and the radially inner surface of the collar, opposite the pin, the spring biasing the pin inwardly through the orifice and into engagement with one of the first cavity and the second cavity at successive intervals of advancement of the mover through the internal bore of the elongate housing, the collar being depressed radially inward, toward the elongate housing, against a bias of the spring, to move the pin out of engagement with one of the first cavity and the second cavity, to enable the mover to be advanced through the internal bore of the elongate housing.

20. The intraocular lens inserter of claim 8, wherein the detent comprises a protrusion that extends outwardly from the mover, further comprising an orifice formed in the elongate housing and sized to engage the protrusion at the at least one predefined interval.

21. The intraocular lens inserter of claim 20, wherein the detent comprises a longitudinally extending component having an outer edge, a distal end, and a proximal end, said protrusion being formed along the outer edge of the longitudinally extending component, extending outwardly from the first cavity, and having a proximal face and a distal face.

22. The intraocular lens inserter of claim 21, wherein the distal face of the protrusion abuts against a portion of the elongate housing to engage the elongate housing at the at least one predefined interval, wherein one portion of the elongate housing comprises the orifice.

23. The intraocular lens inserter of claim 22, wherein said protrusion is forcibly disengaged from the portion of the elongate housing in response to an increased amount of force being applied to advance the mover distally.

24. The intraocular lens inserter of claim 21, wherein the distal end of the longitudinally extending component is mounted to the plunger by a cantilever, so that said protrusion is biased outwardly from the first cavity by the cantilever.

25. The intraocular lens inserter of claim 24, wherein the longitudinally extending component further includes a disengager, which when activated, disengages said protrusion from the orifice to enable withdrawal of the mover proximally from the housing.

26. The intraocular lens inserter of claim 2, further comprising an annular ring disposed around and mounted on the elongate housing to provide a surface for assisting a user in applying force to the proximal end of the mover while grasping the elongate housing.

27. The intraocular lens inserter of claim 2, further comprising a surface area disposed at the proximal end of the plunger, said surface area being disposed such that pressure applied thereto is directed along the longitudinal axis to advance the plunger through the internal bore of the elongate housing.

28. The intraocular lens inserter of claim 3, wherein the elongate housing includes a first section at its distal end that is pivotally mounted to a second section of the elongate housing, so as to pivot to a desired angle relative to the second section, said push rod being sufficiently flexible to readily bend when advancing through the desired angle to controllably expel an artificial lens from a cartridge.

29. The intraocular lens inserter of claim 3, wherein the elongate housing includes a first section at its distal end that forms a desired fixed angle relative to a second section of the elongate housing, said push rod being sufficiently flexible to readily bend when advancing through the desired fixed angle to controllably expel an artificial lens from a cartridge.

30. A method for implanting an artificial intraocular lens into an eye, by advancing the artificial intraocular lens from a cartridge in a controlled manner, comprising the steps of:
(a) advancing the artificial intraocular lens through a distal portion of the cartridge until a detent prevents further advancement beyond a first step;
(b) placing the distal end of the cartridge into the eye;
(c) activating the detent to enable further advancement of the artificial intraocular lens through the distal portion of the cartridge until reaching a second step, wherein further advancement is prevented, thereby providing control that ensures the artificial intraocular lens slowly and controllably exits the distal portion of the cartridge; and
(d) activating the detent to enable further advancement of the artificial intraocular lens through the distal portion of the cartridge until the artificial intraocular lens is fully forced from the distal portion of the cartridge and fully implanted in the eye.

31. The method of claim 30, further comprising the step of preventing the user from advancing the lens from the cartridge too rapidly, if the user depresses the detent to an extent that would avoid stopping the advancement at the first step.

32. The method of claim 30, further comprising the steps of:
(a) positioning the artificial intraocular lens in the cartridge; and
(b) wherein the step of placing the distal end of the cartridge into the eye comprises the step of placing the cartridge with the artificial intraocular lens positioned therein adjacent to an opening in the eye, so that the artificial intraocular lens is able to be readily advanced from the cartridge and into the eye in a controlled manner.

33. The method of claim 30, further comprising the step of placing the cartridge into a cartridge receiver opening of an internal bore of an elongate housing used for introducing the artificial intraocular lens into the eye from the cartridge.

34. The method of claim 33, further comprising the step of pivoting the cartridge relative to a portion of the elongate housing that is held by a user, to achieve a desired angular orientation of the cartridge when inserting the artificial intraocular lens into the eye.

35. An intraocular lens inserter for implanting an artificial intraocular lens into an eye in a controlled manner, comprising:
(a) an elongate housing having an internal bore and including:
(i) a handle portion having a distal end and a proximal end disposed along a longitudinal axis; and
(ii) a cartridge receiver portion having a distal end and a proximal end, the cartridge receiver portion being one of pivotally mounted and fixedly mounted adjacent to the distal end of the handle portion; and
(b) a mover sized to slide within the internal bore of the elongate housing, along the longitudinal axis of the handle portion, the mover having a distal end that is adapted to pass through the cartridge receiver portion and force an artificial intraocular lens therefrom and into an eye, and a proximal end adapted to receive a manual force applied by an operator to advance the mover along the longitudinal axis of the handle portion, to introduce the artificial intraocular lens into an eye.

36. The intraocular lens inserter of claim 35, wherein the mover includes a plunger at its proximal end and an advancer at its distal end, the advancer being sufficiently flexible so that the advancer readily bends when passing from the handle portion and into the cartridge receiver portion and the cartridge receiver portion is pivoted to an acute angle relative to the handle portion.

37. The intraocular lens inserter of claim 36, further comprising a bore reducer disposed in the internal bore of the handle portion, said bore reducer preventing buckling of the advancer within the handle portion, by confining the advancer to minimize its transverse deflection relative to the longitudinal axis of the handle portion.

38. The intraocular lens inserter of claim 36, further comprising a surface area disposed at the proximal end of the plunger, said surface area enabling a force directed along the longitudinal axis to be applied to advance the plunger along the longitudinal axis of the handle portion.

39. The intraocular lens inserter of claim 35, further comprising a spring to provide a force that resists advancing the mover distally through the elongate housing over at least a portion of a path along which the mover advances through the handle portion, when controllably expelling an artificial intraocular lens from a cartridge.

40. The intraocular lens inserter of claim 35, wherein the cartridge receiver portion is fixedly mounted at a desired angular orientation relative to the handle portion, wherein the desired angular orientation is selected to facilitate introducing an artificial intraocular lens into an eye.

41. The intraocular lens inserter of claim 35, wherein the cartridge receiver portion is selectively pivotal to a desired angular orientation relative to the handle portion when being used to introduce an artificial intraocular lens into an eye.

42. The intraocular lens inserter of claim 41, wherein the distal end of the handle portion includes two opposed tabs for pivotally mounting the proximal end of the cartridge receiver portion.

43. The intraocular lens inserter of claim 42, wherein the cartridge receiver portion is pivotal relative to the distal end of the handle portion between at least an angular position in which a cartridge is loadable into the cartridge receiver portion, and a desired angular position at which to introduce an artificial intraocular lens into an eye.

44. The intraocular lens inserter of claim 43, wherein the cartridge receiver portion is pivotal relative to the handle portion to form an angle of about 90 degrees, to enable a cartridge to be loaded into the cartridge receiver portion.

45. The intraocular lens inserter of claim 35, wherein the proximal end of the cartridge receiver portion comprises one of:
(a) a plurality of adjacent flat surfaces;
(b) a plurality of spaced apart protrusions; and
(c) a plurality of spaced apart notches, each flat surface, or protrusion, or notch interacting with the elongate housing to establish a different angular orientation of the cartridge receiving portion relative to the handle portion.

46. The intraocular lens inserter of claim 45, wherein the elongate housing further comprises a sliding block disposed inside the elongate housing adjacent to the distal end of the handle portion and having a distal end and a proximal end.

47. The intraocular lens inserter of claim 46, wherein if the proximal end of the cartridge receiver portion comprises the plurality of adjacent flat surfaces, the distal end of the sliding block abuts at least one flat surface of the proximal end of the cartridge receiver portion to bias the cartridge receiver portion at an angular orientation.

48. The intraocular lens inserter of claim 46, wherein if the proximal end of the cartridge receiver portion comprises the plurality of spaced apart protrusions, the distal end of the sliding block further comprises at least one notch, such that the at least one notch engages at least one protrusion disposed at the proximal end of the cartridge receiver portion, to bias the cartridge receiver portion at an angular orientation.

49. The intraocular lens inserter of claim 46, wherein if the proximal end of the cartridge receiver portion comprises the plurality of spaced apart notches, the distal end of the sliding block further comprises at least one protrusion, such that said at least one protrusion engages at least one notch at the proximal end of the cartridge receiver portion, to bias the cartridge receiver portion at an angular orientation.

50. The intraocular lens inserter of claim 46, further comprising a first spring to provide a force against the sliding block that advances the sliding block distally of the handle portion and against the cartridge receiver portion, such that the cartridge receiver portion is stabilized at a desired angular orientation relative to the handle portion.

51. The intraocular lens inserter of claim 50, further comprising a second spring to provide a force that resists advancing the mover distally through the housing portion over at least a portion of a path along which the mover advances through the handle portion, and which also applies at least a portion of a biasing force applied against the sliding block to bias the sliding block distally of the handle portion and against the cartridge receiver portion.

52. The intraocular lens inserter of claim 35, further comprising means for movably retaining the cartridge receiver portion at a desired angular orientation relative to the handle portion.

53. The intraocular lens inserter of claim 35, wherein the means engage an outer wall of the cartridge receiver portion.
